SEQUENCE LISTING (43 sequences)

SEQ ID NO: 1 - DNA, 18 bp, synthetic construct
  misc_feature 1..18: note = Synthetic construct Mutant protein NS3 F primer
  source 1..18: mol_type = other DNA
gctcctcctttccattgc

SEQ ID NO: 2 - DNA, 20 bp, synthetic construct
  misc_feature 1..20: note = Synthetic construct Mutant protein NS3 R primer
  source 1..20: mol_type = other DNA
ttcttctctttcttggtgcc

SEQ ID NO: 3 - DNA, 20 bp, synthetic construct
  misc_feature 1..20: note = Synthetic construct Mutant protein E2 F primer
  source 1..20: mol_type = other DNA
tgatgatgtgcgtcttagcc

SEQ ID NO: 4 - DNA, 18 bp, synthetic construct
  misc_feature 1..18: note = Synthetic construct Mutant protein E2 R primer
  source 1..18: mol_type = other DNA
atcgtgcctcgaatagcg

SEQ ID NO: 5 - RNA, 11696 nt, unidentified
  misc_feature 1..11696: note = Alphavirus M1 genome
  source 1..11696: mol_type = unassigned RNA
atggcggacgtgtgacatcatcgttcgctctttctaggatcctttgctactccacatagtgagagacaaacaacccaaatgaaggtaaccgtggacgttgaggctgatagcccattccttaaggcccttcagaaggcgtttcccgcctttgaggttgaatcacagcaggtcacaccgaatgaccatgctaacgctagagcattttcgcatctggctactaaactgattgagcaagaggttccaacaggcgtcaccatcctggacgtgggtagtgcacccgcaaggaggttgatgtctgaccacacctaccactgcatctgccccatgaaaagtgcggaagacccagagaggctggcgaattacgctcgaaagctggcgaaagcatcggggactgtgctagacaagaatgtgtccggaaagataacggacctacaagacgtcatggccactccagacttggaatccccgactttttgcctgcacactgacgagacgtgccgcactagggctgaggtcgctgtgtaccaggacgtatacgctgtgcacgcaccgacgtcactgtatcaccaggccatcaaaggtgtcaggacggcgtattggattggattcgacaccactccattcatgttcgaggcactagcgggcgcgtatcctgcgtactcgaccaactgggcagatgagcaagtgctgcaggctcgtaacatcggcctgtgcgcgacaggcctctccgaggggcgtcgcggcaaactctccatcatgagaaagaagtgcttgcgaccgagcgacagagtaatgttttcggtcgggtccaccttgtacaccgagagccgaaagctgctgcgcagctggcatttaccttccgtgtttcacctgaagggtaagaacagttttacctgcaggtgcgacacggtggtgtcatgcgaaggttacgtggtaaagaagatcaccataagcccgggcatatatggaaaaacagtcgattacgcagttacccatcacgcagagggttttctggtgtgtaagatcactgatacagtcagaggagaaagagtctctttcccggtctgtacctatgtgcctgcaaccatatgcgatcagatgacgggtatacttgccaccgacgtgacaccagaggatgcccagaagctcctggttggattgaaccaacgcatagtggtgaatggtaggacgcaaagaaacacaaacacaatgaaaaactatctactgccagtggtagcgcaagcattcagtaaatgggcacgagaggcacgcgcagacatggaggatgaaaaacccctaggcaccagagaacgcacgttgacgtgttgttgcctgtgggcgtttaaaagccacaaaacccacaccatgtataagcggcctgaaactcaaactatcgtcaaagtgccttccacttttgactcctttgtgataccgagcctgaggtcatccagtctttccatggatatcagacggaggatcaaactgctactcagcgcaagaatggcccaaggcctaccatactcaggagaccgcactgaagctcgcgcggcagaagaagaagagaaggaggcgcaggaggcagaacttacgagggcagcgctgccaccgctagtgagcggctcttgtgctgacgatatcgcccaggtggatgtagaggaattaaccttcagagccggagccggggttgtggaaacacccaggaatgccctgaaggttacaccgcaagcacacgaccacctcataggctcctacttgatcctttccccccaaacggtgttgaaaagcgagaagctggcacccatccaccctcttgctgagcaagtcacggtcatgacccactctggaagatccggcagatacccagtcgacaagtacgacggacgggtattgatcccaacaggagcggccatcccagtgagtgagttccaggcactcagcgagagcgcaaccatggtgtacaatgagagggaatttataaatcgcaagctacaccacatagcgctatacgggccagccttgaataccgacgaggaaagctacgaaaaagtgagagctgagagggcagagacagagtatgtgtttgacgtggacaagaaggcatgtatcaagaaggaggaggcatcaggccttgtgttaacaggggacctaatcaatccacctttccacgaattcgcatacgaaggactcaagatccgcccagcagccccgtaccacacgacgatcattggtgtgtttggcgttccaggttcgggcaagtcggctatcattaagaacatggtgacgactcgcgatctggtggccagtggaaagaaggagaactgccaagagatcatgaatgatgtaaagaggcaacgcgggttggacgtgaccgctaggaccgtcgactcaatcttattgaatgggtgcaagaaaggcgtagaaaacctttacgtcgatgaggcgttcgcgtgtcactcgggcactttgctagcgctcatcgcgctggtgagaccgtcaggtaaggtagtactgtgcggcgaccccaagcagtgtggtttcttcaatttgatgcaactgaaggtgcactataaccacaacatttgtacaagggtgctccataagagcatctccagaagatgcactctacctgttacggcgatcgtgtccaccttgcactaccaagggaagatgagaacgacgaaccgatgcaacacccccattcagattgacaccaccggttcttccaaaccagcctcaggagatatcgtgttaacgtgcttccgcggctgggtgaagcaactgcaaatcgactatcgtggacacgaggtgatgaccgcagctgcttcccagggtctgacaaggaaaggcgtgtacgccgtgagacagaaagtgaacgaaaacccactgtactcaccactgtcggagcacgtcaatgtgctgttgacccgaactgaaaaccgactggtgtggaagacactgtcgggtgacccgtggataaaggtgttaaccaatgttccacgtggggatttcagtgcaactttggaggaatggcaagaagaacatgacggtatcatgagagtgttgaacgagcgaccggcggaggttgatccattccaaaacaaggccaaagtgtgctgggcaaaatgtctggtgcaagttcttgagacggccggaatacgtatgacggcagatgaatggaacaccatcttggctttcagagaggacagagcgtactcaccagaagtcgctctcaacgagatttgcactcgttactacggcgttgacctagacagcggcctattctcagcgcagtcagtttccctcttttatgagaacaaccactgggacaacaggcccggaggacgcatgtacgggttcaaccatgaagtagccaggaaatatgcagccaggtttccatttctacgtggcaacatgaactcggggctacaactaaacgtccctgagaggaagctccaaccttttagcgctgaatgcaatatagtcccatccaatcgtcggttaccgcatgctctagtcacaagctatcagcagtgccgtggggagagggtagagtggttgctgaaaaagattccaggtcaccaaatgttacttgtaagtgagtacaacctggtgatacctcacaaaagagtcttctggattgcacctccgcgggtgtcaggcgcggaccgcacgtacgacatggacctagggttacctatggatgcaggccgttacgatctggtattcgtcaacatccatactgagtaccggcaacaccactaccaacaatgcgtcgaccattcaatgcgtctgcagatgctgggaggggattcactacacctgcttagaccaggaggctcgctgctgatgagagcatatggttacgcagacagagtcagcgagatggtggtgacagccctggctaggaaattctcggcgttccgtgtcctgagaccggcgtgtgtgacgagcaacacagaagtgttcctgctgttttctaactttgataacggcagaagagcggtaaccttgcaccaagctaaccagaaacttagctcaatgtatgcctgcaacggactgcacactgctggctgtgcaccgtcatacagggtccgccgcgcagatatatcaggacacggtgaggaagcagtcgtaaatgctgccaatgccaagggtaccgtgagcgatggagtgtgcagggcggtcgctaagaagtggccatcatctttcaaaggggctgcaactccagtcggcacagccaagatgatccgcgcagatggcatgaccgtaatccacgcagtgggaccgaacttctccaccgtaacagaagccgaaggggacagagagctagcggccgcgtaccgagctgtggctagcataattagtaccaacaacataaagagcgtcgcagtaccgctgctatccacaggcaccttttccggcggtaaggacagagtgatgcagtccttgaaccacttattcacggcattggatgccaccgatgcagacgtggttatctactgcagagataaaaactgggaaaagaagattcaggaagccatcgacaggcggacggcaatagagctcgtatccgaagacgtgaccttggaaaccgatctggttagggtacacccggacagttgcttagtcggcagaaatggttacagtgcaactgacggtaaactgtactcttaccttgagggcacgaggttccaccagacggcggtcgacatggctgaaatatcaactttatggccaagactccaagatgctaacgagcagatctgcctgtacgccctaggggagacgatggacagcatacgcactaaatgcccagtagaggacgccgattcgtctacgccgccgaaaacggtaccgtgtctatgtcggtacgcgatgaccgcggagcgggttgccagacttaggatgaataacaccaaaaacatcatcgtgtgctcctcctttccattgccgaagtacaggatagaaggcgtgcagaaggtgaagtgtgaccgagtgctaatttttgaccagaccgtcccgtcactagtaagtcccagaaagtacatacagcagccgccggaacagctggataatgtgagcctgacttctacgacgtcgacgggatccgcatggtcatttccatcggaaacgacctacgaaaccatggaagtcgtagccgaggtacacaccgaacctccgatccctccgcctcgccgacgtagagcagccgtcgcccaacttagacaggatctggaagtcaccgaggagatcgagccgtacgtgatacagcaagcagagatcatggtcatggagagggtcgcgacgacagacatacgcgctatcccagtcccggcacggcgggccattacaatgccagtcccagcccccagtgttcgtaaggtcgctaccgaacctccattagaaccggaagctcctatcccggcaccaagaaagagaagaaccactggcaccacacctccgcataaccccgaggatttcgttcccagggtaccggttgagttaccgtgggagccggaagacctagacatccaattcggtgacttggagccacgccgccggaacaccagggaccgagatgtcagcacaggaatacagttcggtgacatcgactttaaccagtcctgactaggcagggctggcgcgtatatcttttcgtctgacactggcccgggtcacctacagcagaagtccgtaaggcaacatgaattgccatgcgagactctgtatgcccatgaagacgaacgcatatacccgccggcatttgacggagagaaagagaaggtactccaggcaaagatgcagatggccccgacagaagcgaataagagcaggtaccagtcgaggaaagtagagaacatgaaggcattaattgtagaaagactacgcgaaggagcaaagatgtacctccatgagcaaaccgacaaagtacccacgtacaccagcaagtaccctagacctgtgtactcaccatcggtggatgacagcctgagcgatccggatgtggctgtggccgcctgtaactctttcttagaggagaattatccgaccgtggcgaactaccagataaccgatgagtatgacgcttatctggacttggtcgacggctctgaaagctgcctcgacagagctacgttctgcccggccaaactaagatgttaccctaagcaccacgcataccaccaaccacaaatcaggagcgcagtaccttcccctttccaaaacacgttacaaaacgtgctagccgcggccactaaaagaaattgtaatgtcacccaaatgagagaattaccaaccatggactctgcggtgtttaacgtagaaagcttcaaaaaatacgcctgtaccggcgaatattggcaagaatttaaagacaatcctatacggatcaccaccgaaaacataacgacgtacgtggctaaactcaagggtccaaaggctgctgccctttttgccaagacgcataacctggtgccgctccaggaggtgccaatggaccgcttcgtgatggatatgaagagagatgtgaaagttacaccaggcaccaagcataccgaagaaaggcctaaagtgcaagtgattcaagcggcggaaccattggccacggcatatttatgcggaatccacagagagttagtcaggcggctaaaagccgttctgaccccgaacattcacactctgtttgacatgtccgcggaggactttgatgccatcatagcggcacattttcaaccgggagatgctgtactggagacagatatcgcatccttcgataagagccaagacgactccttagcgctaacggcgctaatgcttctggaagacctcggggtcgaccaagaactgctggaccttatcgaagctgcgtttggtgagatcacgagtgtgcatctacctaccggtacaagatttaaattcggtgctatgatgaagtcaggaatgtttcttacactcttcatcaacacgctgctgaacattgtcatagcgtgccgcgtcttacgcgacaaattatcgtcctcggcgtgcgccgccttcataggtgatgacaacatagtgcacggcgtgaggtcagacccgctaatggcagaaaggtgtgcgagttgggtcaacatggaagtaaagatcatcgatgccacaatgtgtgagaaaccaccgtacttttgtggaggattcatcctgtacgacagtgtcgccggtacagcgtgtagggttgcagacccgttaaagaggctgttcaaactcgggaaaccgctcccagcggacgacaaccaggatgaagacagaagaagggcactaaaagacgaaacagttaagtggtcccgcataggattgagagaagaattagacgtggcactgagctcaagataccaagtcagtggcgtcgggaacatcactagagcgatgtccacgctgtctaagaatttgaagtcttttaggaaaataagaggtcccattatacatctgtacggcggtcctaaatagatgcaggattacactacatctaaagaccacgtattacagacaccatgaattacatcccaactcaaaccttttacggacgccgttggcgaccacgcccggcgttccgtccatggcgggtgccgatgcagccggccccacccatgattccagagctgcaaactccgatcgtccaggcccaacagatgcagcagctaatcagtgcagtttctgccctgacgactaagcaaaatggcaaagcaccgaagaagccgaagaaaaagccgcaaaaagcgaaggctaagaaaaacgaacagcaaaagaaaaacgagaacaagaaaccaccacctaagcagaagaatccggctaagaagaagaaaccaggaaaaagggaacgcatgtgcatgaagatagaaaatgattgcatcttcgaggtcaagcttgacggtaaggtcacgggatacgcctgcctagtcggggataaagtgatgaagccggcacacgtcaaaggtgtgatcgacaaccccgacctagcgaagcttacctacaagaaatcgagcaagtatgacctagagtgcgcccagataccggtgcacatgaagtcagatgcttcaaaatacacccatgaaaaaccagaagggcactacaattggcatcacggtgcagtgcagtacagcggtggcaggttcacaatcccgacaggcgcaggtaaaccaggagacagcggccggccgatcttcgacaacaaaggacgtgtggtggccattgtcctgggaggggccaacgaaggagccaggactgccctatctgtcgtgacctggaccaaagacatggtcacacggtacaccccagaaggaacagaagaatggtccgccgccttgatgatgtgcgtcttagccaacgttacattcccatgctcagagcccgcatgtgcaccctgttgctatgaaaaacaaccagaacagacactgaggatgttggaggacaacgtggaccgcccgggctactacgacctgctcgaggccacgatgacgtgtaacaacagtgcacgccaccgtcgcagtgtgacgaaacacttcaatgtctacaaggccacgaaaccgtacctagcgtattgcgcggactgcggagacgggcagttctgttacagcccggtggctatagaaaaaattagggatgaggcttccgatggcatgataaagatccaggtcgcagcgcaaattggcatcaacaaaggaggaacacacgaacacaacaaaatcaggtacatcgccgggcatgacatgaaagaggcaaaccgggattctttacaagtgcatacttccggtgtgtgcgctattcgaggcacgatgggccacttcatcgtggcctactgccctccagggggcgaactaaaggtccagttccaagatgcagaatcgcacacacaggcctgcaaagtgcagtacaaacacgctccggccccagtaggcagagaaaaattcaccgtcaggccccacttcggtatcgaagtgccatgcacaacgtaccagctgactaccgcaccgacggaggaagagatcgacatgcataccccaccggatatcccagacataacgttgctgtcgcagcggtcaggtaacgtaaagatcacagcaggaggaaaaaccatcagatacaactgcacgtgtggtagtggcaacgtgggcaccaccagtagcgacaagactatcaattcgtgcaaaatagcacagtgccacgctgcggtgactaaccacgataagtggcagtacacctcctcgtttgtccctagagccgaccagttgtctcgcaaaggtaaagtgcacgtacccttccctctgaccaactccacatgcagggtgccccttgcacgtgcaccaggtgtcacatacggaaagagagaactgacagtgaaactgcatccagatcatcccacgctgttgacgtaccggagtctaggagcagatccgcgtccgtatgaggagtggatagaccgatacgtcgaacggaccataccggtgaccgaagatgggatagagtacagatggggaaacaacccacccgtgcgcttgtgggcccagctgacaactgaaggcaaaccccatgggtggccgcacgagatcatactctattactatgggctatacccagcagccaccatcgtcgctgtctcagccgcgtgtctcgcagtcgtactatcgctgctggcgtcatgttacatgttcgccactgcacgccgcaagtgcctgaccccatacgccctgacccccggagccgtcgtcccggtaacactaggagtactatgctgcgcaccacgagcgcatgccgcgtcgtttgcggaatctatggcgtatctatgggatgagaatcaaaccctgttttggctggagcttgcaacgccgctcgctgccataatcatacttgtatgctgcctgaagaacctgctttgctgctgcaaaccgctatcttttttagtgttggtgagcctgggaactcccgtcgtaaaatcttacgaacacaccgcaacgatcccgaatgtggtgggattcccgtataaggctcacattgagaggaacggcttctccccgatgaccctacagcttgaggtacttggaaccagcttggaacccacactaaacttagagtacataacctgtgaatacaagacagtcgtgccgtcaccttatatcaagtgctgcgggacatcagaatgcagatccatggagcgccccgactatcaatgccaggtctacacaggagtgtacccatttatgtggggcggcgcatactgcttctgcgacactgagaacacccagctgagtgaagcatacgttgacagatcggacgtatgcaagcacgaccatgccgcggcctacaaggcgcatactgcggcaatgaaagccaccatccgaataagctacgggaacctcaatcagacaacaacggcgttcgtcaacggggagcacacagtgaccgtcggaggcagcaggtttacttttggtccaatctccactgcctggacgcctttcgacaacaagattgtcgtctataagaacgacgtctacaacctggacttcccaccctacgggtcaggacaaccaggaaggtttggagacatccagagcaggacggtagagagcaaggacctgtatgccaacaccgccctcaagttgtcaagaccttcgtccggtactgttcacgtgccttacacacagaccccttctggctttaagtactggataaaagagagaggcacgtcgctgaatgacaaggctccctttggatgcgtaatcaagaccaacccagttagagcagaaaattgcgccgttggcaacatcccagtctccatggacatcccggactccgcgtttacgcgcgtgattgatgcacctgccgtcacaaacctggagtgccaagtggcggtctgcacgcactcatcggacttcggcgggattgcgactctgactttcaaaaccgacaaacccggaaaatgtgctgtccattctcattcgaacgtagccaccatacaggaggcagctgtggacatcaaaacagatggcaagataaccctgcatttctctacagcatcagcatccccggcattcaaggtatctgtgtgcagtgccaaaacgacatgcatggcagcgtgtgagccgccgaaggatcacatcgtcccttatggggcgagccataacaaccaagtttttcctgacatgtctggcacggcaatgacatgggtgcagcgggtagctggcggattcggcgggctaacactcgccgctgtggcagtacttatactggtgacgtgtgtgactatgcgccgctaaccgggaggcttgacataatgtatatatataagcatcatagttttaataaagcatataaataatcaagtagatcaaagggctacctaacccctgaatagtaacaaaacgcaaaatacaaaaacattagttcaaagggccagtaacccctgaatagtaacaaaacataaaaaccaaaaaaaaaaaaaaacagtagttcaaagggctatacaacccctgaatagtaacaaaatacagaaaaaccataaaaattataaaattaactaatctgatcatctaaatttgactaattggaaatagccgaactctacggagatgtaggcgtccgaactccacggagacgtaggacaaaattctgccgaaccccagaccatcggggacgtaggcgtctaatttgtttttttaatattttac

SEQ ID NO: 6 - protein, 534 aa, unidentified
  REGION 1..534: note = Alphavirus M1 NS1 protein
MKVTVDVEADSPFLKALQKAFPAFEVESQQVTPNDHANARAFSHLATKLIEQEVPTGVTILDVGSAPARRLMSDHTYHCICPMKSAEDPERLANYARKLAKASGTVLDKNVSGKITDLQDVMATPDLESPTFCLHTDETCRTRAEVAVYQDVYAVHAPTSLYHQAIKGVRTAYWIGFDTTPFMFEALAGAYPAYSTNWADEQVLQARNIGLCATGLSEGRRGKLSIMRKKCLRPSDRVMFSVGSTLYTESRKLLRSWHLPSVFHLKGKNSFTCRCDTVVSCEGYVVKKITISPGIYGKTVDYAVTHHAEGFLVCKITDTVRGERVSFPVCTYVPATICDQMTGILATDVTPEDAQKLLVGLNQRIVVNGRTQRNTNTMKNYLLPVVAQAFSKWAREARADMEDEKPLGTRERTLTCCCLWAFKSHKTHTMYKRPETQTIVKVPSTFDSFVIPSLRSSSLSMDIRRRIKLLLSARMAQGLPYSGDRTEARAAEEEEKEAQEAELTRAALPPLVSGSCADDIAQVDVEELTFRAGA

SEQ ID NO: 7 - protein, 798 aa, unidentified
  REGION 1..798: note = Alphavirus M1 NS2 protein
GVVETPRNALKVTPQAHDHLIGSYLILSPQTVLKSEKLAPIHPLAEQVTVMTHSGRSGRYPVDKYDGRVLIPTGAAIPVSEFQALSESATMVYNEREFINRKLHHIALYGPALNTDEESYEKVRAERAETEYVFDVDKKACIKKEEASGLVLTGDLINPPFHEFAYEGLKIRPAAPYHTTIIGVFGVPGSGKSAIIKNMVTTRDLVASGKKENCQEIMNDVKRQRGLDVTARTVDSILLNGCKKGVENLYVDEAFACHSGTLLALIALVRPSGKVVLCGDPKQCGFFNLMQLKVHYNHNICTRVLHKSISRRCTLPVTAIVSTLHYQGKMRTTNRCNTPIQIDTTGSSKPASGDIVLTCFRGWVKQLQIDYRGHEVMTAAASQGLTRKGVYAVRQKVNENPLYSPLSEHVNVLLTRTENRLVWKTLSGDPWIKVLTNVPRGDFSATLEEWQEEHDGIMRVLNERPAEVDPFQNKAKVCWAKCLVQVLETAGIRMTADEWNTILAFREDRAYSPEVALNEICTRYYGVDLDSGLFSAQSVSLFYENNHWDNRPGGRMYGFNHEVARKYAARFPFLRGNMNSGLQLNVPERKLQPFSAECNIVPSNRRLPHALVTSYQQCRGERVEWLLKKIPGHQMLLVSEYNLVIPHKRVFWIAPPRVSGADRTYDMDLGLPMDAGRYDLVFVNIHTEYRQHHYQQCVDHSMRLQMLGGDSLHLLRPGGSLLMRAYGYADRVSEMVVTALARKFSAFRVLRPACVTSNTEVFLLFSNFDNGRRAVTLHQANQKLSSMYACNGLHTAGC

SEQ ID NO: 8 - protein, 523 aa, unidentified
  REGION 1..523: note = Alphavirus M1 NS3 protein
APSYRVRRADISGHGEEAVVNAANAKGTVSDGVCRAVAKKWPSSFKGAATPVGTAKMIRADGMTVIHAVGPNFSTVTEAEGDRELAAAYRAVASIISTNNIKSVAVPLLSTGTFSGGKDRVMQSLNHLFTALDATDADVVIYCRDKNWEKKIQEAIDRRTAIELVSEDVTLETDLVRVHPDSCLVGRNGYSATDGKLYSYLEGTRFHQTAVDMAEISTLWPRLQDANEQICLYALGETMDSIRTKCPVEDADSSTPPKTVPCLCRYAMTAERVARLRMNNTKNIIVCSSFPLPKYRIEGVQKVKCDRVLIFDQTVPSLVSPRKYIQQPPEQLDNVSLTSTTSTGSAWSFPSETTYETMEVVAEVHTEPPIPPPRRRRAAVAQLRQDLEVTEEIEPYVIQQAEIMVMERVATTDIRAIPVPARRAITMPVPAPSVRKVATEPPLEPEAPIPAPRKRRTTGTTPPHNPEDFVPRVPVELPWEPEDLDIQFGDLEPRRRNTRDRDVSTGIQFGDIDFNQSLGRAGA

SEQ ID NO: 9 - protein, 611 aa, unidentified
  REGION 1..611: note = Alphavirus M1 NS4 protein
YIFSSDTGPGHLQQKSVRQHELPCETLYAHEDERIYPPAFDGEKEKVLQAKMQMAPTEANKSRYQSRKVENMKALIVERLREGAKMYLHEQTDKVPTYTSKYPRPVYSPSVDDSLSDPDVAVAACNSFLEENYPTVANYQITDEYDAYLDLVDGSESCLDRATFCPAKLRCYPKHHAYHQPQIRSAVPSPFQNTLQNVLAAATKRNCNVTQMRELPTMDSAVFNVESFKKYACTGEYWQEFKDNPIRITTENITTYVAKLKGPKAAALFAKTHNLVPLQEVPMDRFVMDMKRDVKVTPGTKHTEERPKVQVIQAAEPLATAYLCGIHRELVRRLKAVLTPNIHTLFDMSAEDFDAIIAAHFQPGDAVLETDIASFDKSQDDSLALTALMLLEDLGVDQELLDLIEAAFGEITSVHLPTGTRFKFGAMMKSGMFLTLFINTLLNIVIACRVLRDKLSSSACAAFIGDDNIVHGVRSDPLMAERCASWVNMEVKIIDATMCEKPPYFCGGFILYDSVAGTACRVADPLKRLFKLGKPLPADDNQDEDRRRALKDETVKWSRIGLREELDVALSSRYQVSGVGNITRAMSTLSKNLKSFRKIRGPIIHLYGGPK

SEQ ID NO: 10 - protein, 267 aa, unidentified
  REGION 1..267: note = Alphavirus M1 C protein
MNYIPTQTFYGRRWRPRPAFRPWRVPMQPAPPMIPELQTPIVQAQQMQQLISAVSALTTKQNGKAPKKPKKKPQKAKAKKNEQQKKNENKKPPPKQKNPAKKKKPGKRERMCMKIENDCIFEVKLDGKVTGYACLVGDKVMKPAHVKGVIDNPDLAKLTYKKSSKYDLECAQIPVHMKSDASKYTHEKPEGHYNWHHGAVQYSGGRFTIPTGAGKPGDSGRPIFDNKGRVVAIVLGGANEGARTALSVVTWTKDMVTRYTPEGTEEW

SEQ ID NO: 11 - protein, 64 aa, unidentified
  REGION 1..64: note = Alphavirus M1 E3 protein
SAALMMCVLANVTFPCSEPACAPCCYEKQPEQTLRMLEDNVDRPGYYDLLEATMTCNNSARHRR

SEQ ID NO: 12 - protein, 422 aa, unidentified
  REGION 1..422: note = Alphavirus M1 E2 protein
SVTKHFNVYKATKPYLAYCADCGDGQFCYSPVAIEKIRDEASDGMIKIQVAAQIGINKGGTHEHNKIRYIAGHDMKEANRDSLQVHTSGVCAIRGTMGHFIVAYCPPGGELKVQFQDAESHTQACKVQYKHAPAPVGREKFTVRPHFGIEVPCTTYQLTTAPTEEEIDMHTPPDIPDITLLSQRSGNVKITAGGKTIRYNCTCGSGNVGTTSSDKTINSCKIAQCHAAVTNHDKWQYTSSFVPRADQLSRKGKVHVPFPLTNSTCRVPLARAPGVTYGKRELTVKLHPDHPTLLTYRSLGADPRPYEEWIDRYVERTIPVTEDGIEYRWGNNPPVRLWAQLTTEGKPHGWPHEIILYYYGLYPAATIVAVSAACLAVVLSLLASCYMFATARRKCLTPYALTPGAVVPVTLGVLCCAPRAHA

SEQ ID NO: 13 - protein, 61 aa, unidentified
  REGION 1..61: note = Alphavirus M1 6K protein
ASFAESMAYLWDENQTLFWLELATPLAAIIILVCCLKNLLCCCKPLSFLVLVSLGTPVVKS

SEQ ID NO: 14 - protein, 438 aa, unidentified
  REGION 1..438: note = Alphavirus M1 E1 protein
YEHTATIPNVVGFPYKAHIERNGFSPMTLQLEVLGTSLEPTLNLEYITCEYKTVVPSPYIKCCGTSECRSMERPDYQCQVYTGVYPFMWGGAYCFCDTENTQLSEAYVDRSDVCKHDHAAAYKAHTAAMKATIRISYGNLNQTTTAFVNGEHTVTVGGSRFTFGPISTAWTPFDNKIVVYKNDVYNLDFPPYGSGQPGRFGDIQSRTVESKDLYANTALKLSRPSSGTVHVPYTQTPSGFKYWIKERGTSLNDKAPFGCVIKTNPVRAENCAVGNIPVSMDIPDSAFTRVIDAPAVTNLECQVAVCTHSSDFGGIATLTFKTDKPGKCAVHSHSNVATIQEAAVDIKTDGKITLHFSTASASPAFKVSVCSAKTTCMAACEPPKDHIVPYGASHNNQVFPDMSGTAMTWVQRVAGGFGGLTLAAVAVLILVTCVTMRR

SEQ ID NO: 15 - RNA, 11696 nt, unidentified
  misc_feature 1..11696: note = Alphavirus M1 Genome
  source 1..11696: mol_type = unassigned RNA
atggcggacgtgtgacatcatcgttcgctctttctaggatcctttgctactccacatagtgagagacaaacaacccaaatgaaggtaaccgtggacgttgaggctgatagcccattccttaaggcccttcagaaggcgtttcccgcctttgaggttgaatcacagcaggtcacaccgaatgaccatgctaacgctagagcattttcgcatctggctactaaactgattgagcaagaggttccaacaggcgtcaccatcctggacgtgggtagtgcacccgcaaggaggttgatgtctgaccacacctaccactgcatctgccccatgaaaagtgcggaagacccagagaggctggcgaattacgctcgaaagctggcgaaagcatcggggactgtgctagacaagaatgtgtccggaaagataacggacctacaagacgtcatggccactccagacttggaatccccgactttttgcctgcacactgacgagacgtgccgcactagggctgaggtcgctgtgtaccaggacgtatacgctgtgcacgcaccgacgtcactgtatcaccaggccatcaaaggtgtcaggacggcgtattggattggattcgacaccactccattcatgttcgaggcactagcgggcgcgtatcctgcgtactcgaccaactgggcagatgagcaagtgctgcaggctcgtaacatcggcctgtgcgcgacaggcctctccgaggggcgtcgcggcaaactctccatcatgagaaagaagtgcttgcgaccgagcgacagagtaatgttttcggttgggtccaccttgtacaccgagagccgaaagctgctgcgcagctggcatttaccttccgtgtttcacctgaagggtaagaacagttttacctgcaggtgcgacacggtggtgtcatgcgaaggttacgtggtaaagaagatcaccataagcccgggcatatatggaaaaacagtcgattacgcagttacccatcacgcagagggttttctggtgtgtaagatcactgatacagtcagaggagaaagagtctctttcccggtctgtacctatgtgcctgcaaccatatgcgatcagatgacgggtatacttgccaccgacgtgacaccagaggatgcccagaagctcctggttggattgaaccaacgcatagtggtgaatggtaggacgcaaagaaacacaaacacaatgaaaaactatctactgccagtggtagcgcaagcattcagtaaatgggcacgagaggcacgcgcagacatggaggatgaaaaacccctaggcaccagagaacgcacgttgacgtgttgttgcctgtgggcgtttaaaagccacaaaacccacaccatgtataagcggcctgaaactcaaactatcgtcaaagtgccttccacttttgactcctttgtgataccgagcctgaggtcatccagtctttccatggatatcagacggaggatcaaactgctactcagcgcaagaatggcccaaggcctaccatactcaggagaccgcactgaagctcgcgcggcagaagaagaagagaaggaggcgcaggaggcagaacttacgagggcagcgctgccaccgctagtgagcggctcttgtgctgacgatatcgcccaggtggatgtagaggaattaaccttcagagccggagccggggttgtggaaacacccaggaatgccctgaaggttacaccgcaagcacacgaccacctcataggctcctacttgatcctttccccccaaacggtgttgaaaagcgagaagctggcacccatccaccctcttgctgagcaagtcacggtcatgacccactctggaagatccggcagatacccagtcgacaagtacgacggacgggtattgatcccaacaggagcggccatcccagtgagtgagttccaggcactcagcgagagcgcaaccatggtgtacaatgagagggaatttataaatcgcaagctacaccacatagcgctatacgggccagccttgaataccgacgaggaaagctacgaaaaagtgagagctgagagggcagagacagagtatgtgtttgacgtggacaagaaggcatgtatcaagaaggaggaggcatcaggccttgtgttaacaggggacctaatcaatccacctttccacgaattcgcatacgaaggactcaagatccgcccagcagccccgtaccacacgacgatcattggtgtgtttggcgttccaggttcgggcaagtcggctatcattaagaacatggtgacgactcgcgatctggtggccagtggaaagaaggagaactgccaagagatcatgaatgatgtaaagaggcaacgcgggttggacgtgaccgctaggaccgtcgactcaatcttattgaatgggtgcaagaaaggcgtagaaaacctttacgtcgatgaggcgttcgcgtgtcactcgggcactttgctagcgctcatcgcgctggtgagaccgtcaggtaaggtagtactgtgcggcgaccccaagcagtgtggtttcttcaatttgatgcaactgaaggtgcactataaccacaacatttgtacaagggtgctccataagagcatctccagaagatgcactctacctgttacggcgatcgtgtccaccttgcactaccaagggaagatgagaacgacgaaccgatgcaacacccccattcagattgacaccaccggttcttccaaaccagcctcaggagatatcgtgttaacgtgcttccgcggctgggtgaagcaactgcaaatcgactatcgtggacacgaggtgatgaccgcagctgcttcccagggtctgacaaggaaaggcgtgtacgccgtgagacagaaagtgaacgaaaacccactgtactcaccactgtcggagcacgtcaatgtgctgttgacccgaactgaaaaccgactggtgtggaagacactgtcgggtgacccgtggataaaggtgttaaccaatgttccacgtggggatttcagtgcaactttggaggaatggcaagaagaacatgacggtatcatgagagtgttgaacgagcgaccggcggaggttgatccattccaaaacaaggccaaagtgtgctgggcaaaatgtctggtgcaagttcttgagacggccggaatacgtatgacggcagatgaatggaacaccatcttggctttcagagaggacagagcgtactcaccagaagtcgctctcaacgagatttgcactcgttactacggcgttgacctagacagcggcctattctcagcgcagtcagtttccctcttttatgagaacaaccactgggacaacaggcccggaggacgcatgtacgggttcaaccatgaagtagccaggaaatatgcagccaggtttccatttctacgtggcaacatgaactcggggctacaactaaacgtccctgagaggaagctccaaccttttagcgctgaatgcaatatagtcccatccaatcgtcggttaccgcatgctctagtcacaagctatcagcagtgccgtggggagagggtagagtggttgctgaaaaagattccaggtcaccaaatgttacttgtaagtgagtacaacctggtgatacctcacaaaagagtcttctggattgcacctccgcgggtgtcaggcgcggaccgcacgtacgacatggacctagggttacctatggatgcaggccgttacgatctggtattcgtcaacatccatactgagtaccggcaacaccactaccaacaatgcgtcgaccattcaatgcgtctgcagatgctgggaggggattcactacacctgcttagaccaggaggctcgctgctgatgagagcatatggttacgcagacagagtcagcgagatggtggtgacagccctggctaggaaattctcggcgttccgtgtcctgagaccggcgtgtgtgacgagcaacacagaagtgttcctgctgttttctaactttgataacggcagaagagcggtaaccttgcaccaagctaaccagaaacttagctcaatgtatgcctgcaacggactgcacactgctggctgtgcaccgtcatacagggtccgccgcgcagatatatcaggacacggtgaggaagcagtcgtaaatgctgccaatgccaagggtaccgtgagcgatggagtgtgcagggcggtcgctaagaagtggccatcatctttcaaaggggctgcaactccagtcggcacagccaagatgatccgcgcagatggcatgaccgtaatccacgcagtgggaccgaacttctccaccgtaacagaagccgaaggggacagagagctagcggccgcgtaccgagctgtggctagcataattagtaccaacaacataaagagcgtcgcagtaccgctgctatccacaggcaccttttccggcggtaaggacagagtgatgcagtccttgaaccacttattcacggcattggatgccaccgatgcagacgtggttatctactgcagagataaaaactgggaaaagaagattcaggaagccatcgacaggcggacggcaatagagctcgtatccgaagacgtgaccttggaaaccgatctggttagggtacacccggacagttgcttagtcggcagaaatggttacagtgcaactgacggtaaactgtactcttaccttgagggcacgaggttccaccagacggcggtcgacatggctgaaatatcaactttatggccaagactccaagatgctaacgagcagatctgcctgtacgccctaggggagacgatggacagcatacgcactaaatgcccagtagaggacgccgattcgtctacgccgccgaaaacggtaccgtgtctatgtcggtacgcgatgaccgcggagcgggttgccagacttaggatgaataacaccaaaaacatcatcgtgtgctcctcctttccattgccgaagtacaggatagaaggcgtgcagaaggtgaagtgtgaccgagtgctaatttttgaccagaccgtcccgtcactagtaagtcccagaaagtacatacagcagccgccggaacagctggataatgtgagcctgacttctacgacgtcgacgggatccgcatggtcatttccatcggaaacgacctacgaaaccatggaagtcgtagccgaggtacacaccgaacctccgatccctccgcctcgccgacgtagagcagccgtcgcccaacttagacaggatctggaagtcaccgaggagatcgagccgtacgtgatacagcaagcagagatcatggtcatggagagggtcgcgacgacagacatacgcgctatcccagtcccggcacggcgggccattacaatgccagtcccagcccccagtgttcgtaaggtcgctaccgaacctccattagaaccggaagctcctatcccggcaccaagaaagagaagaaccactggcaccacacctccgcataaccccgaggatttcgttcccagggtaccggttgagttaccgtgggagccggaagacctagacatccaattcggtgacttggagccacgccgccggaacaccagggaccgagatgtcagcacaggaatacagttcggtgacatcgactttaaccagtcctgactaggcagggctggcgcgtatatcttttcgtctgacactggcccgggtcacctacagcagaagtccgtaaggcaacatgaattgccatgcgagactctgtatgcccatgaagacgaacgcatatacccgccggcatttgacggagagaaagagaaggtactccaggcaaagatgcagatggccccgacagaagcgaataagagcaggtaccagtcgaggaaagtagagaacatgaaggcattaattgtagaaagactacgcgaaggagcaaagatgtacctccatgagcaaaccgacaaagtacccacgtacaccagcaagtaccctagacctgtgtactcaccatcggtggatgacagcctgagcgatccggaagtggctgtggccgcctgtaactctttcttagaggagaattatccgaccgtggcgaactaccagataaccgatgagtatgatgcttatctggacttggtcgacggctctgaaagctgcctcgacagagctacgttctgcccggccaaactaagatgttaccctaagcaccacgcataccaccaaccacaaatcaggagcgcagtaccttcccctttccaaaacacgttacaaaacgtgctagccgcggccactaaaagaaattgtaatgtcacccaaatgagagaattaccaaccatggactctgcggtgtttaacgtagaaagcttcaaaaaatacgcctgtaccggcgaatattggcaagaatttaaagacaatcctatacggatcaccaccgaaaacataacgacgtacgtggctaaactcaagggtccaaaggctgctgccctttttgccaagacgcataacctggtgccgctccaggaggtgccaatggaccgcttcgtgatggatatgaagagagatgtgaaagttacaccaggcaccaagcataccgaagaaaggcctaaagtgcaagtgattcaagcggcggaaccattggccacggcatatttatgcggaatccacagagagttagtcaggcggctaaaagccgttctgaccccgaacattcacactctgtttgacatgtcggcggaggactttgatgccatcatagcggcacattttcaaccgggagatgctgtactggagacagatatcgcatccttcgataagagccaagacgactccttagcgctaacggcgctaatgcttctggaagacctcggggtcgaccaagaactgctggaccttatcgaagctgcgtttggtgagatcacgagtgtgcatctacctaccggtacaagatttaaattcggtgctatgatgaagtcaggaatgtttcttacactcttcatcaacacgctgctgaacattgtcatagcgtgccgcgtcttacgcgacaaattatcgtcctcggcgtgcgccgccttcataggtgatgacaacatagtgcacggcgtgaggtcagacccgctaatggcagaaaggtgtgcgagttgggtcaacatggaagtaaagatcatcgatgccacaatgtgtgagaaaccaccgtacttttgtggaggattcatcctgtacgacagtgtcgccggtacagcgtgtagggttgcagacccgttaaagaggctgttcaaactcgggaaaccgctcccagcggacgacaaccaggatgaagacagaagaagggcactaaaagacgaaacagttaagtggtcccgcataggattgagagaagaattagacgtggcactgagctcaagataccaagtcagtggcgtcgggaacatcactagagcgatgtccacgctgtctaagaatttgaagtcttttaggaaaataagaggtcccattatacatctgtacggcggtcctaaatagatgcaggattacactacatctaaagaccacgtattacagacaccatgaattacatcccaactcaaaccttttacggacgccgttggcgaccacgcccggcgttccgtccatggcgggtgccgatgcagccggccccacccatgattccagagctgcaaactccgatcgtccaggcccaacagatgcagcagctaatcagtgcagtttctgccctgacgactaagcaaaatggcaaagcaccgaagaagccgaagaaaaagccgcaaaaagcgaaggctaagaaaaacgaacagcaaaagaaaaacgagaacaagaaaccaccacctaagcagaagaatccggctaagaagaagaaaccaggaaaaagggaacgcatgtgcatgaagatagaaaatgattgcatcttcgaggtcaagcttgacggtaaggtcacgggatacgcctgcctagtcggggataaagtgatgaagccggcacacgtcaaaggtgtgatcgacaaccccgacctagcgaagcttacctacaagaaatcgagcaagtatgacctagagtgcgcccagataccggtgcacatgaagtcagatgcttcaaaatacacccatgaaaaaccagaagggcactacaattggcatcacggtgcagtgcagtacagcggtggcaggttcacaatcccgacaggcgcaggtaaaccaggagacagcggccggccgatcttcgacaacaaaggacgtgtggtggccattgtcctgggaggggccaacgaaggagccaggactgccctatctgtcgtgacctggaccaaagacatggtcacacggtacaccccagaaggaacagaagaatggtccgccgccttgatgatgtgcgtcttagccaacgttacattcccatgctcagagcccgcatgtgcaccctgttgctatgaaaaacaaccagaacagacactgaggatgttggaggacaacgtggaccgcccgggctactacgacctgctcgaggccacgatgacgtgtaacaacagtgcacgccaccgtcgcagtgtgacggaacacttcaatgtctacaaggccacgaaaccgtacctagcgtattgcgcggactgcggagacgggcagttctgttacagcccggtggctatagaaaaaattagggatgaggcttccgatggcatgataaagatccaggtcgcagcgcaaattggcatcaacaaaggaggaacacacgaacacagcaaaatcaggtacatcgccgggcatgacatgaaagaggcaaaccgggattctttacaagtgcatacttccggtgtgtgcgctattcgaggcacgatgggccacttcatcgtggcctactgccctccagggggcgaactaaaggtccagttccaagatgcagaatcgcacacacaggcctgcaaagtgcagtacaaacacgctccggccccagtaggcagagaaaaattcaccgtcaggccccacttcggtatcgaagtgccatgcacaacgtaccagctgactaccgcaccgacggaggaagagatctacatgcataccccaccggatatcccagacataacgttgctgtcgcagcggtcaggtaacgtaaagatcacagcaggaggaaaaaccatcagatacaactgcacgtgtggtagtggcaacgtgggcaccaccagtagcgacaagactatcaattcgtgcaaaatagcacagtgccacgctgcggtgactaaccacgataagtggcagtacacctcctcgtttgtccctagagccgaccagttgtctcgcaaaggtaaagtgcacgtacccttccctctgaccaactccacatgcagggtgccccttgcacgtgcaccaggtgtcacatacggaaagagagaactgacagtgaaactgcatccagatcatcccacgctgttgacgtaccggagtctaggagcagatccgcgtccgtatgaggagtggatagaccgatacgtcgaacggaccataccggtgaccgaagatgggatagagtacagatggggaaacaacccacccgtgcgcttgtgggcccagctgacaactgaaggcaaaccccatgggtggccgcacgagatcatactctattactatgggctatacccagcagccaccatcgtcgctgtctcagccgcgtgtctcgcagtcgtactatcgctgctggcgtcatgttacatgttcgccactgcacgccgcaagtgcctgaccccatacgccctgacccccggagccgtcgtcccggtaacactaggagtactatgctgcgcaccacgagcgcatgccgcgtcgtttgcggaatctatggcgtatctatgggatgagaatcaaaccctgttttggctggagcttgcaacgccgctcgctgccataatcatacttgtatgctgcctgaagaacctgctttgctgctgcaaaccgctatcttttttagtgttggtgagcctgggaactcccgtcgtaaaatcttacgaacacaccgcaacgatcccgaatgtggtgggattcccgtataaggctcacattgagaggaacggcttctccccgatgaccctacagcttgaggtacttggaaccagcttggaacccacactaaacttagagtacataacctgtgaatacaagacagtcgtgccgtcaccttatatcaagtgctgcgggacatcagaatgcagatccatggagcgccccgactatcaatgccaggtctacacaggagtgtacccatttatgtggggcggcgcatactgcttctgcgacactgagaacacccagctgagtgaagcatacgttgacagatcggacgtatgcaagcacgaccatgccgcggcctacaaggcgcatactgcggcaatgaaagccaccatccgaataagctacgggaacctcaatcagacaacaacggcgttcgtcaacggggagcacacagtgaccgtcggaggcagcaggtttacttttggtccaatctccactgcctggacgcctttcgacaacaagattgtcgtctataagaacgacgtctacaacctggacttcccaccctacgggtcaggacaaccaggaaggtttggagacatccagagcaggacggtagagagcaaggacctgtatgccaacaccgccctcaagttgtcaagaccttcgtccggtactgttcacgtgccttacacacagaccccttctggctttaagtactggataaaagagagaggcacgtcgctgaatgacaaggctccctttggatgcgtaatcaagaccaacccagttagagcagaaaattgcgccgttggcaacatcccagtctccatggacatcccggactccgcgtttacgcgcgtgattgatgcacctgccgtcacaaacctggagtgccaagtggcggtctgcacgcactcatcggacttcggcgggattgcgactctgactttcaaaaccgacaaacccggaaaatgtgctgtccattctcattcgaacgtagccaccatacaggaggcagctgtggacatcaaaacagatggcaagataaccctgcatttctctacagcatcagcatccccggcattcaaggtatctgtgtgcagtgccaaaacgacatgcatggcagcgtgtgagccgccgaaggatcacatcgtcccttatggggcgagccataacaaccaagtttttcctgacatgtctggcacggcaatgacatgggtgcagcgggtagctggcggattcggcgggctaacactcgccgctgtggcagtacttatactggtgacgtgtgtgactatgcgccgctaaccgggaggcttgacataatgtatatatataagcatcatagttttaataaagcatataaataatcaagtagatcaaagggctacctaacccctgaatagtaacaaaacgcaaaatacaaaaacattagttcaaagggccagtaacccctgaatagtaacaaaacataaaaaccaaaaaaaaaaaaaaacagtagttcaaagggctatacaacccctgaatagtaacaaaatacagaaaaaccataaaaattataaaattaactaatctgatcatctaaatttgactaattggaaatagccgaactctacggagatgtaggcgtccgaactccacggagacgtaggacaaaattctgccgaaccccagaccatcggggacgtaggcgtctaatttgtttttttaatattttac

SEQ ID NO: 16 - protein, 534 aa, unidentified; identical to SEQ ID NO: 6
  REGION 1..534: note = Alphavirus M1 NS1 protein

SEQ ID NO: 17 - protein, 798 aa, unidentified; identical to SEQ ID NO: 7
  REGION 1..798: note = Alphavirus M1 NS2 protein

SEQ ID NO: 18 - protein, 523 aa, unidentified; identical to SEQ ID NO: 8
  REGION 1..523: note = Alphavirus M1 NS3 protein

SEQ ID NO: 19 - protein, 611 aa, unidentified
  REGION 1..611: note = Alphavirus M1 NS4 protein
YIFSSDTGPGHLQQKSVRQHELPCETLYAHEDERIYPPAFDGEKEKVLQAKMQMAPTEANKSRYQSRKVENMKALIVERLREGAKMYLHEQTDKVPTYTSKYPRPVYSPSVDDSLSDPEVAVAACNSFLEENYPTVANYQITDEYDAYLDLVDGSESCLDRATFCPAKLRCYPKHHAYHQPQIRSAVPSPFQNTLQNVLAAATKRNCNVTQMRELPTMDSAVFNVESFKKYACTGEYWQEFKDNPIRITTENITTYVAKLKGPKAAALFAKTHNLVPLQEVPMDRFVMDMKRDVKVTPGTKHTEERPKVQVIQAAEPLATAYLCGIHRELVRRLKAVLTPNIHTLFDMSAEDFDAIIAAHFQPGDAVLETDIASFDKSQDDSLALTALMLLEDLGVDQELLDLIEAAFGEITSVHLPTGTRFKFGAMMKSGMFLTLFINTLLNIVIACRVLRDKLSSSACAAFIGDDNIVHGVRSDPLMAERCASWVNMEVKIIDATMCEKPPYFCGGFILYDSVAGTACRVADPLKRLFKLGKPLPADDNQDEDRRRALKDETVKWSRIGLREELDVALSSRYQVSGVGNITRAMSTLSKNLKSFRKIRGPIIHLYGGPK

SEQ ID NO: 20 - protein, 267 aa, unidentified; identical to SEQ ID NO: 10
  REGION 1..267: note = Alphavirus M1 C protein

SEQ ID NO: 21 - DNA, 49 bp, synthetic construct
  misc_feature 1..49: note = Synthetic construct 5173
  source 1..49: mol_type = other DNA
ttgaccagaccgtcccgtcactagtaagtcccagaaagtacatacagca

SEQ ID NO: 22 - DNA, 23 bp, synthetic construct
  misc_feature 1..23: note = Synthetic construct 5230
  source 1..23: mol_type = other DNA
acttccagggtttcgtaggtcgt

SEQ ID NO: 23 - DNA, 48 bp, synthetic construct
  misc_feature 1..48: note = Synthetic construct 4907
  source 1..48: mol_type = other DNA
actccaagatgctaacgagcagatctgcctgtacgccctaggggagac

SEQ ID NO: 24 - DNA, 23 bp, synthetic construct
  misc_feature 1..23: note = Synthetic construct 5298
  source 1..23: mol_type = other DNA
acgacctacgaaaccctggaagt

SEQ ID NO: 25 - DNA, 58 bp, synthetic construct
  misc_feature 1..58: note = Synthetic construct 7104
  source 1..58: mol_type = other DNA
ctgacttcatcatagcaccgaatttaaatcttgtaccggtaggtagatgcacactcgt

SEQ ID NO: 26 - DNA, 54 bp, synthetic construct
  misc_feature 1..54: note = Synthetic construct 9420
  source 1..54: mol_type = other DNA
cgggctactacgacctgctcgaggccacgatgacgtgtaacaacagtgcacgcc

SEQ ID NO: 27 - DNA, 58 bp, synthetic construct
  misc_feature 1..58: note = Synthetic construct 9510
  source 1..58: mol_type = other DNA
ttgtagacattgaagtggttcgtcacactgcgacggtggcgtgcactgttgttacacg

SEQ ID NO: 28 - DNA, 25 bp, synthetic construct
  misc_feature 1..25: note = Synthetic construct 9486
  source 1..25: mol_type = other DNA
tgacgaaccacttcaatgtctacaa

SEQ ID NO: 29 - DNA, 45 bp, synthetic construct
  misc_feature 1..45: note = Synthetic construct 10521
  source 1..45: mol_type = other DNA
ggtttgccttcagttgtcagctgggcccacaagcgcacgggtggg

SEQ ID NO: 30 - protein, 64 aa, unidentified; identical to SEQ ID NO: 11
  REGION 1..64: note = Alphavirus M1 E3 protein

SEQ ID NO: 31 - protein, 422 aa, unidentified
  REGION 1..422: note = Alphavirus M1 E2 protein
SVTEHFNVYKATKPYLAYCADCGDGQFCYSPVAIEKIRDEASDGMIKIQVAAQIGINKGGTHEHSKIRYIAGHDMKEANRDSLQVHTSGVCAIRGTMGHFIVAYCPPGGELKVQFQDAESHTQACKVQYKHAPAPVGREKFTVRPHFGIEVPCTTYQLTTAPTEEEIYMHTPPDIPDITLLSQRSGNVKITAGGKTIRYNCTCGSGNVGTTSSDKTINSCKIAQCHAAVTNHDKWQYTSSFVPRADQLSRKGKVHVPFPLTNSTCRVPLARAPGVTYGKRELTVKLHPDHPTLLTYRSLGADPRPYEEWIDRYVERTIPVTEDGIEYRWGNNPPVRLWAQLTTEGKPHGWPHEIILYYYGLYPAATIVAVSAACLAVVLSLLASCYMFATARRKCLTPYALTPGAVVPVTLGVLCCAPRAHA

SEQ ID NO: 32 - protein, 61 aa, unidentified; identical to SEQ ID NO: 13
  REGION 1..61: note = Alphavirus M1 6K protein

SEQ ID NO: 33 - protein, 438 aa, unidentified; identical to SEQ ID NO: 14
  REGION 1..438: note = Alphavirus M1 E1 protein

SEQ ID NO: 34 - DNA, 48 bp, synthetic construct
  misc_feature 1..48: note = Synthetic construct NS3 protein consensus sequence
  source 1..48: mol_type = other DNA
tccatcggaaacgacctacgaaaccctggaagtcgtagccgaggtaca

SEQ ID NO: 35 - DNA, 48 bp, synthetic construct
  misc_feature 1..48: note = Synthetic construct rM1-E2M-NS3seq.abi
  source 1..48: mol_type = other DNA
tccatcggaaacgacctacgaaaccatggaagtcgtagccgaggtaca

SEQ ID NO: 36 - DNA, 48 bp, synthetic construct; identical to SEQ ID NO: 34
  misc_feature 1..48: note = Synthetic construct C6V1-NS3sep.abi
  source 1..48: mol_type = other DNA

SEQ ID NO: 37 - DNA, 48 bp, synthetic construct; identical to SEQ ID NO: 34
  misc_feature 1..48: note = Synthetic construct rM1-N3E2M-NS3seq.abi
  source 1..48: mol_type = other DNA

SEQ ID NO: 38 - DNA, 48 bp, synthetic construct; identical to SEQ ID NO: 34
  misc_feature 1..48: note = Synthetic construct rM1-NS3M-NS3seq.abi
  source 1..48: mol_type = other DNA

SEQ ID NO: 39 - DNA, 48 bp, synthetic construct
  misc_feature 1..48: note = Synthetic construct E2 protein consensus sequence
  source 1..48: mol_type = other DNA
gtgcacgccaccgtcgcagtgtgacgaaccacttcaatgtctacaagg

SEQ ID NO: 40 - DNA, 48 bp, synthetic construct; identical to SEQ ID NO: 39
  misc_feature 1..48: note = Synthetic construct rM1-E2M-E2seq.abi
  source 1..48: mol_type = other DNA

SEQ ID NO: 41 - DNA, 48 bp, synthetic construct; identical to SEQ ID NO: 39
  misc_feature 1..48: note = Synthetic construct rM1-N3E2M-E2seq.abi
  source 1..48: mol_type = other DNA

SEQ ID NO: 42 - DNA, 48 bp, synthetic construct
  misc_feature 1..48: note = Synthetic construct C6V1-E2seq.abi
  source 1..48: mol_type = other DNA
gtgcacgccaccgtcgcagtgtgacggaccacttcaatgtctacaagg

SEQ ID NO: 43 - DNA, 48 bp, synthetic construct
  misc_feature 1..48: note = Synthetic construct rM1-NS3M-E2seq.abi
  source 1..48: mol_type = other DNA
gtgcacgccaccgtcgcagtgtgacgaaacacttcaatgtctacaagg

(19) United States
(12) Patent Application Publication
(10) Pub. No.: US 2026/0125657 A1
(43) Pub. Date: May 7, 2026

(54) M1 VIRUS MUTANT AND USE THEREOF

(71) Applicant: GUANGZHOU VIROTECH PHARMACEUTICAL CO., LTD., Guangdong (CN)

(72) Inventors: Guangmei YAN, Guangzhou (CN); Yuan LIN, Guangzhou (CN); Li GUO, Guangzhou (CN); Ziqing LIN, Guangzhou (CN); Guang'en WU, Guangzhou (CN)

(21) Appl. No.: 19/429,209

(22) Filed: Dec. 22, 2025

Related U.S. Application Data
(63) Continuation of application No. 17/595,889, now Pat. No. 12,529,037.

(30) Foreign Application Priority Data
May 31, 2019 (CN) 201910472439.7

(51) Int. Cl. C12N 7/00; A61K 35/768; A61K 45/06; A61P 35/00; C07K 14/005; C07K 14/11; C12N 15/86

(52) U.S. Cl. CPC C12N 7/00; A61K 35/768; A61K 45/06; A61P 35/00; C07K 14/005; C07K 14/11; C12N 15/86; C12N 2770/36121; C12N 2770/36122; C12N 2770/36143

(57) ABSTRACT

Provided is an M1 virus. Further provided are a series of uses of said virus. The uses include, but are not limited to, viral vectors, anti-tumor agents, and pharmaceutical compositions. Said virus can effectively inhibit the growth of various tumor cells, and at the same time, has tumor targeting properties and is non-toxic to normal cells. Said virus can be administrated by means of intravenous injection, having operational convenience.

### CROSS REFERENCE TO RELATED APPLICATIONS

This application is a continuation of U.S. application Ser. No. 17/595,889, filed Nov. 29, 2021, which is a national stage application of International Appl. PCT/CN2020/093642, filed May 31, 2020, and which claims foreign priority from Chinese Appl. No. 201910472439.7, filed May 31, 2019, all of which are hereby incorporated by reference.

### TECHNICAL FIELD

The present disclosure belongs to the field of biological medicines, and particularly relates to an M1 virus mutant and a use thereof.

### BACKGROUND ART

Genic and epigenetic accumulated changes in normal cells drive normal cells to change into malignant tumor cells. This complex pathological process determines the diversity of mechanisms in the formation, maintenance, and metastasis of different tumors (1-3). Surgical excision, chemotherapy, and radiotherapy are conventional methods for treating tumors, the surgical excision easily leads to tumor recurrence, and the chemotherapy and the radiotherapy induce great toxic and side effects (4). Targeted therapies and tumor immunotherapy, including IL-2 control, adoptive cell therapy, and regulation of immune checkpoints such as PD-1, that have emerged in recent years have achieved certain effects in clinical treatment. However, the targeted therapies are prone to drug resistance, and the immunotherapy has a low response rate and may lead to serious immune-associated adverse events (5, 6). Therefore, it is urgent to develop more novel anticancer therapies which have low toxicity and high efficacy and difficultly cause drug resistance.

Oncolytic viruses not only kill tumor cells, but also alert a host's immune system of the presence of cancer. Virotherapy is a therapy based on characteristics of the oncolytic viruses that are more likely to attack cancer tissues rather than healthy tissues. In 2005, the China Food and Drug Administration approved the marketing of the first oncolytic virotherapy with a trade name of Oncorine. This is a genetically modified virus that preferentially attack tumor cells and has been used to treat head and neck cancers. In 2015, the U.S. Food and Drug Administration (FDA) approved a melanoma therapy T-VEC that uses genetically modified herpes viruses, and was approved in Australia and the European Union in the following year. In addition, there are also some oncolytic viruses under research. Although, in recent decades, the virotherapy has made great progress, it still faces numerous difficulties.

First, the efficacy is concerned. The oncolytic viruses have a limited anti-tumor effect or anti-tumor spectrum, many oncolytic viruses cannot well inhibit or kill tumor cells, and need to be used in combination with other chemotherapeutic drugs or immune checkpoint inhibitors, or used as a supplement to radiotherapy. For example, an M1 virus disclosed in a Chinese invention patent application No. 201410425510.3, when used as an anti-tumor drug, has a significantly effect on colorectal cancer, liver cancer, bladder cancer, and breast cancer, has a lower effect on pancreatic cancer, nasopharyngeal carcinoma, prostate cancer, and melanoma, has a much lower effect on a glioma, cervical cancer, and lung cancer, and has the lowest effect on stomach cancer.

Second, the safety is concerned. Certain viruses themselves are dangerous to human bodies, and the dangerous viruses need to be modified and attenuated before being used in the virotherapy. Even if the oncolytic viruses are modified and attenuated, they still can become “escaping viruses”, i.e. viruses that change again or are bound to existing pathogens in a patient's body after release to rapidly infect healthy tissues.

Furthermore, the delivery of viruses is concerned. That is, how to deliver viruses to a lesion. In most of the existing oncolytic virotherapies such as the melanoma therapy T-VEC approved by the U.S. Food and Drug Administration (FDA), the oncolytic viruses are injected into a tumor tissue. However, lesions and micrometastases of many solid tumors cannot be injected directly, or non-solid tumors such as hematological tumors are distributed throughout a body without a fixed injection site. It is difficult to adopt the existing virotherapies to treat these types of tumors.

Therefore, the development of oncolytic viruses still poses great challenges.

Alphaviruses belong to the Togaviridae family, which are a class of single positive-stranded RNA viruses with an envelope structure. It is reported in literatures that a Chikungunya virus belonging to the Alphavirus is a pathogenic virus for humans and highly toxic, and causes fever, rash, arthritis, and even fatal encephalitis after infection (7, 8). It is reported that another virus, i.e. a Venezuelan equine encephalitis virus, belonging to the Alphavirus can be used as a vector to transduce dendritic cells so as to treat tumors (9), but this encephalitis virus has caused fever, convulsions, abortion, and even death in humans (10).

M1 virus (Alphavirus M1) belongs to the Alphavirus (Alphavirus). It was isolated from Culex mosquitoes on Hainan Island, China in 1964, and a method for isolating the virus has been disclosed in a literature (11). The M1 virus belongs to Getah-like viruses and its homology with the Getah virus is up to 97.8% (12). In 2008, the whole genome of an M1 strain was sequenced (13). According to the patent CN 201410425510.3, the M1 virus has an oncolytic effect, but the anti-tumor spectrum and anti-tumor strength of existing wild-type M1 viruses need to be improved.

### SUMMARY OF THE INVENTION

In some embodiments, an M1 virus is provided. An amino acid residue corresponding to the 358th site of the NS3 protein of the M1 virus is not M; and/or an amino acid residue corresponding to the 4th site of the envelope protein E2 is not E or K.

In some embodiments, the amino acid residue corresponding to the 358th site of the NS3 protein (nonstructural protein 3) of the M1 virus is: G, A, L, I, V, P, S, Q, T, C, N, F, Y, W, D, E, K, R or H; and/or the amino acid residue corresponding to the 4th site of the envelope protein E2 (envelope protein 2) is: M, G, A, L, I, V, P, S, Q, T, C, N, F, Y, W, D, R or H.

In some embodiments, an amino acid sequence of the NS3 protein comprised in the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 8 or SEQ ID NO: 18; and/or an amino acid sequence of the E2 protein comprised in the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 12 or SEQ ID NO: 31. In some embodiments, an M1 virus is provided. An amino acid residue corresponding to the 358th site of the NS3 protein encoded by a nucleotide sequence of the M1 virus is not M; and/or an amino acid residue corresponding to the 4th site of the E2 protein is not E or K.

In some embodiments, the nucleotide sequence of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an M1 sequence shown as SEQ ID NO: 5 or SEQ ID NO: 15 or GenBank Accession No. EU015061.1 or GenBank Accession No. EF011023.1 or CCTCC V201423.

In some embodiments, preferably, the amino acid residue corresponding to the 358th site of the NS3 protein is: G, A, L, I, V, P, S, Q, T, C, N, F, Y, W, D, E, K, R or H; and/or the amino acid residue corresponding to the 4th site of the E2 protein is: M, G, A, L, I, V, P, S, Q, T, C, N, F, Y, W, D, R or H.

In some embodiments, the M1 virus has a mutation relative to a wild-type M1 virus or a pseudo-wild-type M1 virus.

In some embodiments, the M1 virus has a mutation relative to an M1 virus having a sequence shown as SEQ ID NO: 5 or an M1 virus having a sequence shown as SEQ ID NO: 15.

In some embodiments, the mutation is: M358G, M358A, M358L, M358I, M358V, M358P, M358S, M358Q, M358T, M358C, M358N, M358F, M358Y, M358W, M358D, M358E, M358K, M358R or M358H on the NS3 protein; and/or K4M, K4G, K4A, K4L, K4I, K4V, K4P, K4S, K4Q, K4T, K4C, K4N, K4F, K4Y, K4W, K4D, K4R, K4H, E4M, E4G, E4A, E4L, E4I, E4V, E4P, E4S, E4Q, E4T, E4C, E4N, E4F, E4Y, E4W, E4D, E4R or E4H on the E2 protein.

In some embodiments, the M1 virus is obtained by the mutation of an amino acid residue corresponding to the 358th site of the NS3 protein of the M1 virus having the sequence shown as SEQ ID NO: 5 into: G, A, L, I, V, P, S, Q, T, C, N, F, Y, W, D, E, K, R or H; and/or the mutation of an amino acid residue at the 4th site of the E2 into: M, G, A, L, I, V, P, S, Q, T, C, N, F, Y, W, D, R or H.

In some embodiments, the M1 virus is obtained by the mutation of an amino acid residue M corresponding to the 358th of the NS3 protein of the M1 virus having the sequence shown as SEQ ID NO: 15 into L; and/or by the mutation of an amino acid residue E corresponding to the 4th site of the E2 protein into D.

In some embodiments, a nucleotide sequence comprising any M1 virus described above is provided.

In some embodiments, an amino acid sequence corresponding to the NS3 protein of an M1 virus is provided. An amino acid residue corresponding to the 358th site of the amino acid sequence is not M.

In some embodiments, preferably, the amino acid residue corresponding to the 358th site is: G, A, L, I, V, P, S, Q, T, C, N, F, Y, W, D, E, K, R or H.

In some embodiments, the amino acid sequence corresponding to the NS3 of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 8 or SEQ ID NO: 18.

In some embodiments, an amino acid sequence corresponding to the E2 protein of an M1 virus is further provided. An amino acid residue corresponding to the 4th site of the amino acid sequence is not E or K.

In some embodiments, the amino acid residue corresponding to the 4th site is M, G, A, L, I, V, P, S, Q, T, C, N, F, Y, W, D, R or H.

In some embodiments, the amino acid sequence corresponding to the E2 protein of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 12 or SEQ ID NO: 31.

In some embodiments, a nucleotide sequence for encoding the amino acid sequence corresponding to the NS3 protein of the M1 virus is further provided.

In some embodiments, a nucleotide sequence for encoding the amino acid sequence corresponding to the E2 protein of the M1 virus is further provided.

In some embodiments, a vector is further provided. The vector comprises a nucleic acid for encoding the E2 protein and/or the NS3 protein of an M1 virus; an amino acid residue corresponding to the 358th site of the NS3 protein is not M; and an amino acid residue corresponding to the 4th site of the E2 protein is not E or K.

In some embodiments, the amino acid residue corresponding to the 358th site of the NS3 protein is: G, A, L, I, V, P, S, Q, T, C, N, F, Y, W, D, E, K, R or H.

In some embodiments, the amino acid residue corresponding to the 4th site of the E2 protein is: M, G, A, L, I, V, P, S, Q, T, C, N, F, Y, W, D, R or H.

In some embodiments, an amino acid sequence of the NS3 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 8 or SEQ ID NO: 18.

In some embodiments, an amino acid sequence of the E2 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 12 or SEQ ID NO: 31.

In some embodiments, the vector further comprises a coding sequence of the NS1 protein, the NS2 protein, the NS4 protein, the C protein, the E3 protein, the 6K protein, and/or the E1 protein of the M1 virus.

In some embodiments, an amino acid sequence of the NS1 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 6 or SEQ ID NO: 16.

In some embodiments, an amino acid sequence of the NS2 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 7 or SEQ ID NO: 17.

In some embodiments, an amino acid sequence of the NS4 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 9 or SEQ ID NO: 19.

In some embodiments, an amino acid sequence of the C protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 10 or SEQ ID NO: 20.

In some embodiments, an amino acid sequence of the E3 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 11 or SEQ ID NO: 30.

In some embodiments, an amino acid sequence of the 6K protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 13 or SEQ ID NO: 32.

In some embodiments, an amino acid sequence of the E1 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity (Identity) with an amino acid sequence shown as SEQ ID NO: 14 or SEQ ID NO: 33.

In some embodiments, the vector further comprises exogenous genes relative to the M1 virus.

In some embodiments, the exogenous genes express anti-tumor-associated molecules.

In some embodiments, the vector is selected from viruses.

In some embodiments, the vector is selected from a retrovirus, a Newcastle disease virus, a rabies virus, a vesicular stomatitis virus, a Maraba virus, an alphavirus, a Newcastle disease virus, a reovirus, an adenovirus, an adeno-associated virus, a herpes simplex virus, a vaccinia virus, and a measles virus.

In some embodiments, the vector is selected from plasmids.

In some embodiments, a vector is provided. The vector comprises the nucleotide sequence described above.

In some embodiments, the vector is selected from plasmids.

In some embodiments, a virus vector is provided. The virus is any M1 virus described above.

In some embodiments, the vector is inserted with exogenous genes.

In some embodiments, the exogenous genes express anti-tumor-associated molecules.

In some embodiments, a use of the M1 virus, or the prepared nucleotide sequence, or the amino acid sequence of the NS3 protein of the M1 virus, or the amino acid sequence of the E2 protein of the M1 virus, or the nucleotide sequence corresponding to the NS3 protein or the E2 protein, or the vector, or the virus vector in the preparation of an anti-tumor drug is further provided.

In some embodiments, an anti-tumor agent is further provided. The anti-tumor agent comprises the M1 virus, or the nucleotide sequence, or the amino acid sequence of the NS3 protein of the M1 virus, or the amino acid sequence of the E2 protein of the M1 virus, or the nucleotide sequence, or the vector, or the virus vector.

In some embodiments, a composition is further provided. The composition comprises an effective amount of the M1 virus, or the nucleotide sequence, or the amino acid sequence of the NS3 protein of the M1 virus, or the amino acid sequence of the E2 protein of the M1 virus, or the vector, or the virus vector, and a pharmaceutically acceptable carrier.

In some embodiments, the composition further comprises an immune checkpoint inhibitor.

In some embodiments, the composition further comprises a chemotherapeutic agent.

Of course, in some embodiments, the M1 virus may also optionally not include the immune checkpoint inhibitor and/or the chemotherapeutic agent. In an embodiment of the present disclosure, in a case without any other anti-cancer agent (e.g. a chemotherapeutic agent, an immune checkpoint inhibitor, and other existing substances or tools that have a tumor inhibitory effect), the M1 virus has a high tumor inhibitory effect when used alone.

In some embodiments, the composition comprises 10¹ virus particles or PFUs.

In some embodiments, the composition comprises 10¹ to 10³⁰ virus particles or PFUs.

In some embodiments, the compositions comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² virus particles or PFUs.

In some embodiments, the composition comprises 2×10⁶ virus particles or PFUs.

In some embodiments, the composition is used to resist a tumor.

In some embodiments, a dosage form of the anti-tumor agent or the composition is selected from: an injection, a tablet, a capsule, a kit, and a patch.

In some embodiments, the dosage form is an injection.

In some embodiments, in the use, or the anti-tumor agent, or the composition, the tumor is selected from a solid tumor and a hematological tumor.

In some embodiments, the solid tumor is selected from one or more of liver cancer, colorectal cancer, bladder cancer, breast cancer, cervical cancer, prostate cancer, a glioma, melanoma, pancreatic cancer, nasopharyngeal carcinoma, lung cancer, stomach cancer, adrenocortical carcinoma, accessory renal cortical carcinoma, anal cancer, appendix cancer, astrocytoma, atypical teratoma, a rhabdoid tumor, basal cell carcinoma, cholangiocarcinoma, bladder cancer, bone cancer, a brain tumor, a bronchial tumor, Burkitt lymphoma, a carcinoid tumor, a heart tumor, cholangiocarcinoma, chordoma, carcinoma of large intestine, craniopharyngioma, ductal carcinoma in situ, a germ tumor, endometrial cancer, ependymoma, esophageal cancer, olfactory neuroblastoma, a intracranial germ Cell tumor, an extragonadal germ cell tumor, ocular cancer carcinoma of fallopian tube, carcinoma of gallbladder, head and neck cancer, hypopharyngeal cancer, Kaposi's sarcoma, renal carcinoma, Langerhans cell histiocytosis, laryngeal cancer, lip cancer, oral cancer, Merkel cell carcinoma, malignant mesothelioma, multiple endocrine neoplasia syndrome, mycosis fungoides, carcinoma of nasal cavity and nasal sinuses, neuroblastoma, non-small cell lung cancer, ovarian cancer, a pancreatic neuroendocrine tumor, an islet cell tumor, papillomatosis, paraganglioma, carcinoma of nasal sinuses and nasal cavity, parathyroid carcinoma, carcinoma of penis, throat cancer, a pituitary tumor, pleuropulmonary blastoma, primary peritoneal carcinoma, retinoblastoma, a salivary gland tumor, sarcoma, Sezary syndrome, skin cancer, small cell lung cancer, carcinoma of small intestine, soft tissue sarcoma, squamous cell carcinoma, testicular cancer, thymoma and thymic cancer, thyroid cancer, urethral cancer, uterine cancer, endometrium and uterine sarcoma, vaginal cancer, a vascular tumor, vulvar cancer, and solitary myeloma.

In some embodiments, the hematological tumor is selected from one or more of B-cell acute lymphoblastic leukemia (BALL), T-cell acute lymphoblastic leukemia (TALL), acute lymphoblastic leukemia (ALL), chronic myelogenous leukemia (CML), chronic lymphocytic leukemia (CLL), B-cell promyelocytic leukemia, blastic plasmacytoid dendritic cell neoplasm, Burkitt lymphoma, diffuse large B-cell lymphoma, follicular lymphoma, hairy cell leukemia, small cell or large cell-follicular lymphoma, MALT lymphoma, mantle cell lymphoma, marginal zone lymphoma, multiple myeloma, non-Hodgkin lymphoma, plasmablastic lymphoma, plasmacytoid dendritic cell neoplasm, Waldenstrom macroglobulinemia, and pre-leukemia.

In some embodiments, the tumor is selected from one or more of liver cancer, colorectal cancer, bladder cancer, breast cancer, cervical cancer, prostate cancer, a glioma, melanoma, pancreatic cancer, nasopharyngeal carcinoma, lung cancer, and stomach cancer.

In some embodiments, the obtained M1 virus can effectively treat a variety of tumors including: liver cancer, colorectal cancer, bladder cancer, breast cancer, cervical cancer, prostate cancer, a glioma, melanoma, pancreatic cancer, nasopharyngeal carcinoma, lung cancer, and stomach cancer. In vitro experiments prove that the M1 virus of the present disclosure can induce the apoptosis of a variety of tumor cells; and in vivo experiments prove that the M1 virus of the present disclosure significantly inhibits the growth of liver cancer and colorectal cancer in vivo. No matter in vivo or in vitro, the M1 virus of the present disclosure has higher efficacy compared with the wild-type virus in general.

In some embodiments, the obtained M1 virus has good selectivity and safety. Cell experiment results show that the M1 virus of the present disclosure can selectively kill tumor cells and is non-toxic to normal cells, which indicates that the M1 of the present disclosure is targeted to tumors; animal experiment results show that after being injected into the tail vein, the M1 virus of the present disclosure does not affect the weight and mental state of the nude mouse and is not distributed to normal organs, which indicates that the M1 virus of the present disclosure is safe.

In some embodiments, the obtained M1 virus significantly inhibits the growth of tumors when administrated by intratumor or intravenous injection. Compared to intratumor injection of existing commercially available oncolytic viruses, this administration needs to be carried out by specially trained doctors or nurses, has low patient acceptance, and is not suitable for deep organ tumors and micrometastasis. However, the M1 virus of the present disclosure can be administrated by intravenous injection for treatment, which indicates that the M1 virus of the present disclosure is more convenient and practicable in clinical uses and has a broader use range.

In the present disclosure, unless the context requires otherwise, the words “comprise” and “include” should be understood as including the described steps or elements or collections of steps and elements, but do not exclude any other steps or elements or collections of steps and elements; that is, the words are open limitations.

For example, in some embodiments, the M1 virus “comprises mutations of . . . ”, the “comprise” refers to that the mutations may also include other mutations (especially a silent mutation) in addition to the described mutations (e.g. of the amino acid residue at the 358th site of the nonstructural protein NS3 and/or the amino acid residue at the 4th of the structural protein E2), such as mutations that do not affect functions of the virus; or some mutations that improve certain abilities or reduce the toxicity or improve the stability of the virus without affecting and interfering basic functions of the mutant M1.

In some embodiments, the M1 virus has double-site mutations: M358L on the NS3 protein, and any one of K4N, K4D, E4N, or E4D on the E2 protein. It refers to that the mutant has and only has these mutations, and does not have other mutations.

In some embodiments, a wild-type M1 virus corresponding to the M1 virus is a virus with a Accession No. CCTCC V201423 (specifically described in a Chinese patent 104814984A).

In some embodiments, a genomic sequence of the wild-type M1 virus corresponding to the M1 virus has at least 95%, at least 96%, at least 97%, at least 98%, at least 99%, at least 99.5% or at least 99.8% identity with a genomic sequence of the virus with a Accession No. CCTCC V201423 (described in the Chinese patent 104814984A).

In some embodiments, a wild-type M1 virus corresponding to the M1 virus is shown as GenBank Accession No. EU015061.1 or EF011023.1 (subject to the information on the application date/priority date).

In some embodiments, a genomic sequence of the wild-type M1 virus corresponding to the M1 virus has at least 95%, at least 96%, at least 97%, at least 98%, at least 99%, at least 99.5% or at least 99.8% identity with a genomic sequence of GenBank Accession No. EU015061.1 or EF011023.1 (subject to the information on the application date/priority date) or a virus reported by Zhai Y G (13), and is regarded as a virus probably derived from the same strain as CCTCC V201423.

In some embodiments, the protein described above is isolated polypeptide.

In some embodiments, the nucleic acid described above is isolated polynucleotide.

In some embodiments, the M1 virus described above is an isolated virus.

### BRIEF DESCRIPTION OF THE DRAWINGS

FIG. 1 shows nucleotide sequencing of mutant sites of 4 mutant viruses (rM1-NS3M, rM1-E2M, rM1-N3E2M, and rM1-c6v1). FIG. 1, part A, shows nucleotide sequencing of amino acid residues near the 358th site of the NS3 protein. A nucleotide sequence at this site of a wild-type is CCA, and a corresponding amino acid is M; a nucleotide sequence of the mutant is CCC, and a corresponding amino acid is L; and FIG. 1, part B, shows nucleotide sequencing of amino acid residues near the 4th site of the E2 protein. A nucleotide sequence at this site of the wild-type is AAA or GAA, and a corresponding amino acid is K or E; and a nucleotide sequence of the mutant is AAC or GAC, and a corresponding amino acid is N or D.

FIG. 2 shows enhanced oncolytic effects of site-directed mutant viruses (rM1-NS3M, rM1-E2M, and rM1-N3E2M) on an HCT 116 cell line;

FIG. 2, part A, shows the cell morphology of the HCT 116 cells infected in 1 MOI for 48 h; FIG. 2, part B, shows comparison of survival rates of the cells infected with rM1-WT, rM1-NS3M, rM1-E2M, and rM1-N3E2M viruses in 0.001 to 10 MOI for 72 h by an MTT method.

FIG. 3 shows safety and efficacy tests of an rM1-N3E2M virus on an animal model. A nude mouse subcutaneously bears a tumor HCT116 and is administrated with the virus by intravenous injection 14 days after the tumor inoculation; the volume of the tumor and the weight of the nude mouse are repeatedly measured and subjected to statistics by ANOVA, and when *p<0.05, the result has statistical significance. FIG. 3, part A, shows growth changes of the tumor in the subcutaneous tumor-bearing nude mouse model, and FIG. 3, part B, shows weight changes of the nude mouse in the subcutaneous tumor-bearing nude mouse model.

FIG. 4 shows rM1-N3E2M virus infection, the expression of an apoptosis index Cl-casp3 is up-regulated in a tumor tissue, and the expression of a cell proliferation index Ki67 is down-regulated. After subcutaneous tumor-bearing nude mouse models are treated with rM1-WT and rM1-N3E2M for 3 days, tumor tissues are dissected, and expressions of Cl-casp3 and Ki67 are detected by means of immunohistochemical staining.

FIG. 5 shows that an rM1-N3E2M virus has no toxic lesion in normal organs and tissues.

FIG. 6 shows safety and efficacy experiment results of an M1-c6v1 virus on an animal model: a C57 BL/6 mouse subcutaneously bears a tumor B16-F10 and is administrated with the virus by intravenous injection 11 days after the tumor inoculation; the volume of the tumor and the weight of the nude mouse are repeatedly measured and subjected to statistics by ANOVA, and when *p<0.05, the result has statistical significance. FIG. 6, part A, shows growth changes of the tumor in the subcutaneous tumor-bearing mouse model, and FIG. 6, part B, shows weight changes of the nude mouse in the subcutaneous tumor-bearing mouse model.

FIGS. 7A to 7Z show sequencing of mutant sites of various site-directed mutant strains of an M1 virus: FIG. 7A shows rM1-WT (E2-4K) (AAA); FIG. 7B shows a mutant strain (E2-4L, AAA→CTG); FIG. 7C shows a mutant strain (E2-4I, AAA→ATT); FIG. 7D shows a mutant strain (E2-4V, AAA→GTG); FIG. 7E shows a mutant strain (E2-4S, AAA→AGC); FIG. 7F shows a mutant strain (E2-4C, AAA→TGC); FIG. 7G shows a mutant strain (E2-4L, AAA→CTG); FIG. 7H shows a mutant strain (E2-4D, AAA→GAT); FIG. 7I shows rM1-WT (NS3-358M, ATG); FIG. 7J shows a mutant strain (NS3-358G, ATG→GGC); FIG. 7K shows a mutant strain (NS3-358A, ATG→GCG); FIG. 7L shows a mutant strain (NS3-358L, ATG→CTG); FIG. 7M shows a mutant strain (NS3-358I, ATG→ATT); FIG. 7N shows a mutant strain (NS3-358V, ATG→GTG); FIG. 7O shows a mutant strain (NS3-358P, ATG→CCG); FIG. 7P shows a mutant strain (NS3-358S, ATG→AGC); FIG. 7Q shows a mutant strain (NS3-358Q, ATG→CAG); FIG. 7R shows a mutant strain (NS3-358T, ATG→ACC); FIG. 7S shows a mutant strain (NS3-358C, ATG→TGC); FIG. 7T shows a mutant strain (NS3-358N, ATG→AAC); FIG. 7U shows a mutant strain (NS3-358F, ATG→TTT); FIG. 7V shows a mutant strain (NS3-358Y, ATG→TAT); FIG. 7W shows a mutant strain (NS3-358D, ATG→GAT); FIG. 7X shows a mutant strain (NS3-358K, ATG→AAA); FIG. 7Y shows a mutant strain (NS3-358R, ATG→CGT); and FIG. 7Z shows a mutant strain (NS3-358H, ATG→CAT).

FIGS. 8A to 8Z show kill curves of various site-directed (single-point mutant) mutant strains of an M1 virus to HCT 116: FIG. 8A shows a kill curve of rM-WT (E2-4K); FIG. 8B shows a kill curve of a mutant strain E2-4L; FIG. 8C shows a kill curve of a mutant strain E2-4I; FIG. 8D shows a kill curve of a mutant strain E2-4V; FIG. 8E shows a kill curve of a mutant strain E2-4S; FIG. 8F shows a kill curve of a mutant strain E2-4C; FIG. 8G shows a kill curve of mutant strain E2-4M; FIG. 8H shows a kill curve of a mutant strain E2-4D; FIG. 8I shows a kill curve of rM-WT (NS3-358M); FIG. 8J shows a kill curve of a mutant strain NS3-358G; FIG. 8K shows a kill curve of a mutant strain NS3-358A; FIG. 8L shows a kill curve of a mutant strain NS3-358L; FIG. 8M shows a kill curve of a mutant strain NS3-358I; FIG. 8N shows a kill curve of a mutant strain NS3-358V; FIG. 8O shows a kill curve of a mutant strain NS3-358P; FIG. 8P shows a kill curve of a mutant strain NS3-358S; FIG. 8Q shows a kill curve of a mutant strain NS3-358Q; FIG. 8R shows a kill curve of a mutant strain NS3-358T; FIG. 8S shows a kill curve of a mutant strain NS3-358C; FIG. 8T shows a kill curve of a mutant strain NS3-358N; FIG. 8U shows a kill curve of a mutant strain NS3-358F; FIG. 8V shows a kill curve of a mutant strain NS3-358Y; FIG. 8W shows a kill curve of a mutant strain NS3-358D; FIG. 8X shows a kill curve of a mutant strain NS3-358K; FIG. 8Y shows a kill curve of a mutant strain NS3-358R; and FIG. 8Z shows a kill curve of a mutant strain NS3-358H.

### DETAILED DESCRIPTION OF THE INVENTION

The technical solutions of the present disclosure will be further described below with reference to specific embodiments, which are not intended to limit the scope of protection of the present disclosure. Some non-essential modifications and adjustments made by others based on the concept of the present disclosure shall fall within the scope of protection of the present disclosure.

### Definition

Unless otherwise defined, all technical and scientific terms used in the present disclosure have the same meanings as commonly understood by those of ordinary skill in the art to which the present disclosure belongs.

Unless otherwise specified, the mutation of the present disclosure is: a natural mutation, a mandatory mutation or a selective mutation, and includes but is not limited to gene modification, and sequence increase or deletion or partial substitution.

It should be noted that in the present disclosure, the “mutation” includes an artificial mutation (induced mutation or genetic engineering) at a specific site of a wild-type virus strain. It is well known in the art that the wild-type and the mutant are relative to each other, and wild-type virus strains themselves have differences in nucleotide or amino acid residue sequences.

The term “and/or” as used herein refers to and covers any and all possible combinations of one or more of the associated listed items. When used in a list of two or more items, the term “and/or” means that any of the listed items can be used separately, or any combination of two or more of the listed items can be used. For example, if a composition, a combination or a structure is described as including (or comprising) components A, B, C, and/or D, the composition may only comprise A, only comprise B, only comprise C, only comprise D, comprise a combination of A and B, comprise a combination of A and C, comprise a combination of A and D, comprise a combination of B and C, comprise a combination of B and D, comprise a combination of C and D, comprise a combination of A, B, and C, comprise a combination of A, B, and D, comprise a combination of A, C, and D, comprise a combination of B, C, and D, or comprise a combination of A, B, C, and D.

Here, for example, M358G of the NS3 protein refers to an M358G mutation of an amino acid residue at the 358th of the NS3 protein of a M1 virus, and the amino acid residue mutates from M into G; and for example, “M358A” refers to an M358A mutation of the amino acid residue at the 358th site of the NS3 protein of the M1 virus, and the amino acid residue mutates from M into A. The other is the same.

Here, for example, K4M of the E2 protein refers to a K4M mutation of an amino acid residue at the 4th of the E2 protein of the M1 virus, and the amino acid residue mutates from K into M; and for example, K4A of the E2 protein refers to a K4A mutation of the amino acid residue at the 4th site of the E2 protein of the M1 virus, and the amino acid residue mutates from K into A. The other is the same.

TABLE 1
List of amino acids

| name | Abbreviation | | Structural formula |
|---|---|---|---|
| Glycine | Gly | G | 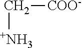 |
| Alanine | Ala | A | 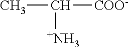 |
| Leucine | Leu | L | 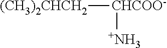 |
| Isoleucine | Ile | I | 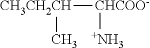 |
| Valine | Val | V | 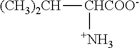 |
| Proline | Pro | P | 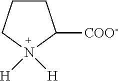 |
| Serine | Ser | S | 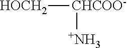 |
| Glutamine | Gln | Q | 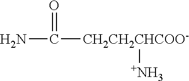 |
| Threonine | Thr | T | 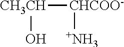 |
| Cysteine | Cys | C | 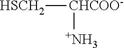 |
| Asparagine | Asn | N | 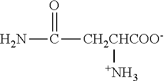 |
| Methionine | Met | M | 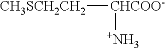 |
| Phenyl- alanine | Phe | F | 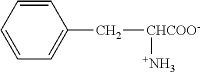 |
| Tyrosine | Tyr | Y | 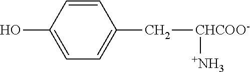 |
| Tryptophan | Trp | W | 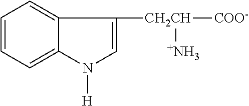 |
| Aspartic acid | Asp | D | 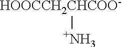 |
| Glutamic acid | Glu | E | 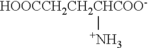 |
| Lysine | Lys | K | 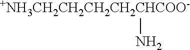 |
| Arginine | Arg | R | 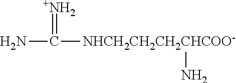 |
| Histidine | His | H | 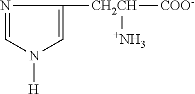 |

NS3 protein: nonstructural protein 3.

E2 protein: envelope protein 2.

Here, the number of various sequences is shown in Table 27 below:

TABLE 27

| | Sequence name (220) | Sequence No. |
|---|---|---|
| PCR primer | Mutant protein NS3 F primer | (SEQ ID NO: 1) |
| | Mutant protein NS3 R primer | (SEQ ID NO: 2) |
| | Mutant protein E2 F primer | (SEQ ID NO: 3) |
| | Mutant protein E2 R primer | (SEQ ID NO: 4) |
| M1 virus artificially | Genome | (SEQ ID NO: 5) |
| synthesized accord- | NS1 protein | (SEQ ID NO: 6) |
| ing to a sequencing | NS2 protein | (SEQ ID NO: 7) |
| result of CCTCC | NS3 protein | (SEQ ID NO: 8) |
| V201423 | NS4 protein | SEQ ID NO: 9) |
| | C protein | (SEQ ID NO: 10) |
| | E3 protein | (SEQ ID NO: 11) |
| | E2 protein | (SEQ ID NO: 12) |
| | 6K protein | (SEQ ID NO: 13) |
| | E1 protein | (SEQ ID NO: 14) |
| M1-c6 virus | Genome | (SEQ ID NO: 15) |
| | NS1 protein | (SEQ ID NO: 16) |
| | NS2 protein | (SEQ ID NO: 17) |
| | NS3 protein | (SEQ ID NO: 18) |
| | NS4 protein | (SEQ ID NO: 19) |
| | C protein | (SEQ ID NO: 20) |
| | E3 protein | (SEQ ID NO: 30) |
| | E2 protein | (SEQ ID NO: 31) |
| | 6K protein | (SEQ ID NO: 32) |
| | E1 protein | (SEQ ID NO: 33) |
| Site-directed | 5173 | (SEQ ID NO: 21) |
| mutation primer | 5230 | (SEQ ID NO: 22) |
| | 4907 | (SEQ ID NO: 23) |
| | 5298 | (SEQ ID NO: 24) |
| | 7104 | (SEQ ID NO: 25) |
| | 9420 | (SEQ ID NO: 26) |
| | 9510 | (SEQ ID NO: 27) |
| | 9486 | (SEQ ID NO: 28) |
| | 10521 | (SEQ ID NO: 29) |

Wild-type M1 virus: it refers to a virus individual obtained in nature, and its corresponding genome is a wild-type genome. The wild-type genome may have diversity at certain specific sites. For example, an amino acid residue at the 4th site of the E2 protein of the known wild-type M1 virus is E or K.

Pseudo-wild-type M1 virus: it refers to an M1 virus having a sequence that does not coincide exactly with that of the known wild-type M1 virus and having biological characteristics that do not significantly differ from those of the known wild-type M1 virus.

In some embodiments, the wild-type M1 virus, for example, may be a M1 virus with a Accession No. CCTCC V201423 or a monoclonal virus M1-c6 virus, or, for example, may be EU015061.1 or EF011023.1.

For example, in some embodiments, mutations may occur at one or more of the following sites on the basis of the wild-type: on amino acid residues at the 2nd site and/or the 786th site of the nonstructural protein NS2; on amino acid residues at the 30th site and/or the 393rd site of the nonstructural protein NS3; on an amino acid residue at the 381st site of the nonstructural protein NS3; on an amino acid residue at the 154th site of the structural protein C; and on an amino acid residue at the 246th site of the structural protein E2.

rM1-WT: it refers to an M1 virus having a genome sequence shown as SEQ ID NO: 5, and it comprises an NS1 protein sequence shown as SEQ ID NO: 6; an NS2 protein sequence shown as SEQ ID NO: 7; an NS3 protein sequence shown as SEQ ID NO: 8; an NS4 protein sequence shown as SEQ ID NO: 9; a C protein sequence shown as SEQ ID NO: 10; an E3 protein sequence shown as SEQ ID NO: 11; an E2 protein sequence shown as SEQ ID NO: 12; a 6K protein sequence shown as SEQ ID NO: 13; and an E1 protein sequence shown as SEQ ID NO: 14. An amino acid residue at the 358th site of the NS3 protein comprised in the M1 virus is M, and an amino acid residue at the 4th of the E2 protein is K.

rM1-NS3M: a recombinant M1 virus obtained by an M358L mutation (i.e. M mutates into L) of an amino acid residue at the 358h site of the NS3 protein on the basis of rM1-WT.

rM1-E2M: a recombinant M1 virus obtained by a K4N mutation (i.e. K mutates into N) of an amino acid residue at the 4th site of the E2 protein on the basis of rM1-WT.

rM1-N3E2M: a recombinant M1 virus obtained by an M358L mutation (i.e. M mutates into L) of an amino acid residue at the 358th site of the NS3 protein on the basis of rM1-WT and a K4N mutation (i.e. K mutates into N) of an amino acid residue at the 4th site of the E2 protein.

M1-c6: it refers to an M1 virus having a genome sequence shown as SEQ ID NO: 15, and it comprises an NS1 protein sequence shown as SEQ ID NO: 16; an NS2 protein sequence shown as SEQ ID NO: 17; an NS3 protein sequence shown as SEQ ID NO: 18; an NS4 protein sequence shown as SEQ ID NO: 19; a C protein sequence shown as SEQ ID NO: 20; an E3 protein sequence shown as SEQ ID NO: 30; an E2 protein sequence shown as SEQ ID NO: 31; a 6K protein sequence shown as SEQ ID NO: 32; and an E1 protein sequence shown as SEQ ID NO: 33. An amino acid residue at the 358th site of the NS3 protein of the M1 virus is M, and an amino acid residue at the 4th of the E2 protein is E.

M1-c6v1: a recombinant M1-c6 virus obtained by an M358L mutation (i.e. M mutates into L) of an amino acid residue at the 358th site of the NS3 protein on the basis of M1-c6 and an E4D mutation (i.e. E mutates into D) of an amino acid residue at the 4th site of the E2 protein.

Amino acid residue at the 358th site of the NS3 protein: in the currently known wild-type M1 virus, this site is ranked 358th in the NS3 protein. A sequence of 3 amino acid residues of the upstream of N-terminal is YET, and a sequence of 3 amino acid residues of the downstream of C-terminal is EVV. The number “358” should not be regarded as an absolute limitation, and whether a specific amino acid residue (e.g. a variant amino acid residue) is located at this site should be determined based on a functional domain or motif where the amino acid residue is located. For example, it is not excluded that in sequences of other pseudo-wild type M1 viruses, this site is not necessarily ranked 358th in the primary structure of the NS3 protein. At this time, “358” should not be regarded as an absolute limitation, and whether the specific amino acid residue belongs to the amino acid residue at the 358th site of the NS3 protein of the present disclosure should be determined based on a functional domain or motif where the amino acid residue is located.

Amino acid residue at the 4th site of the E2 protein: in the currently known wild-type M1 virus, this site is ranked 4th in the E2 protein. A sequence of 3 amino acid residues of the upstream of N-terminal is SVT, and a sequence of 3 amino acid residues of the downstream of C-terminal is HFN. The number “4” should not be regarded as an absolute limitation, and whether a specific amino acid residue (e.g. a variant amino acid residue) is located at this site should be determined based on a functional domain or motif where the amino acid residue is located. For example, it is not excluded that in sequences of other pseudo-wild type M1 viruses, this site is not necessarily ranked 4th in the primary structure of the E2 protein. At this time, “4” should not be regarded as an absolute limitation, and whether the specific amino acid residue belongs to the amino acid residue at the 4th site of the E2 protein of the present disclosure should be determined based on a functional domain or motif where the amino acid residue is located.

Pharmaceutically acceptable carrier: it refers to a molecular entity or a composition that do not produce allergic or similar adverse reactions when administered to humans. The pharmaceutically acceptable carriers include any and all solvents, dispersion media, intermedia, coatings, diluents, antibacterial agents, antifungal agents, isotonic agents, absorption delaying agents, buffers, carrier solutions, suspensions, colloids, etc. The use of these media and reagents for active substances of drugs is well known in the art. The pharmaceutically acceptable carrier is expected to be used in a therapeutic composition except in a case where any conventional medium or agent is incompatible with an active ingredient.

Immune checkpoint inhibitor: it refers to molecules integrally or partially reducing, inhibiting, interfering or regulating one or more checkpoint proteins. The checkpoint proteins regulate and control the activation and functions of T cells. The known checkpoint proteins include, for example, CTLA-4 and ligands CD80 and CD86 thereof; and PD1 and ligands PDL1 and PDL2 thereof (Pardoll, Nature Reviews Cancer 12:252-264, 2012). These proteins are responsible for the co-stimulation or inhibition of interactions of the T-cell response. The immune checkpoint proteins regulate and control and maintain their tolerance and the duration and range of the physiological immune response. The immune checkpoint inhibitors include antibodies or are derived from antibodies.

Chemotherapeutic agent: a compound that can be used to treat cancer.

Treatment: it refers to alleviating symptoms, temporarily or permanently eliminating the cause of symptoms, or preventing or slowing down symptoms of a specified disease or disorder.

Effective amount: it refers to the quantity of an alphavirus or a proteasome inhibitor used in the present disclosure that is required by a desired therapeutic effect. Required precise amount varies depending on objects and the following factors: treated species, age and general conditions of a treated object, the severity of a treated disease, an administrated particular agent, an administration method, etc. However, for a given situation, those of ordinary skill in the art can adjust the dosage of the pharmaceutical composition of the present disclosure according to the severity of symptoms, the frequency of recurrence, and the physiological response to a therapeutic regimen.

### Example 1 Construction and Identification of M1 Site-Directed Mutant Virus Strains

### Materials:

1. Cloning: TOP10 competent cells, plasmid micro-extraction kits, DNA product recovery kits; Gibson Assembly Master Mix; Phanta Max Super-Fidelity DNA Polymerase.
2. Restriction endonucleases SpeI, SwaI, XhoI, ApaI, and XbaI.
3. Site-directed mutation primers shown in Table 2:

TABLE 2
List of site-directed mutation primers

| Name | Sequence |
|---|---|
| 5173 | TTGACCAGACCGTCCCGTCACTAGTAAGTCCCAGAAAGTACATAC |
| | AGCA (SEQ ID NO: 21) |
| 5230 | ACTTCCAGGGTTTCGTAGGTCGT (SEQ ID NO: 22) |
| 4907 | ACTCCAAGATGCTAACGAGCAGATCTGCCTGTACGCCCTAGGGGA |
| | GAC (SEQ ID NO: 23) |
| 5298 | ACGACCTACGAAACCCTGGAAGT (SEQ ID NO: 24) |
| 7104 | CTGACTTCATCATAGCACCGAATTTAAATCTTGTACCGGTAGGTA |
| | GATGCACACTCGT (SEQ ID NO: 25) |
| 9420 | CGGGCTACTACGACCTGCTCGAGGCCACGATGACGTGTAACAACA |
| | GTGCACGCC (SEQ ID NO: 26) |
| 9510 | TTGTAGACATTGAAGTGGTTCGTCACACTGCGACGGTGGCGTGCA |
| | CTGTTGTTACACG (SEQ ID NO: 27) |
| 9486 | TGACGAACCACTTCAATGTCTACAA (SEQ ID NO: 28) |
| 10521 | GGTTTGCCTTCAGTTGTCAGCTGGGCCCACAAGCGCACGGGTGGG |
| | (SEQ ID NO: 29) |

4. Full-length genome plasmids of the wild-type M1 virus: pBR-M1-WT is a plasmid vector comprising an M1 full-genome that has a sequence shown as SEQ ID NO: 5. A full-genome is synthesized according to the sequence shown as SEQ ID NO: 5, and the M1 sequence is cloned between ClaI and EcoRI cleavage sites of a pBR322 vector (added with multiple cloning sites) by a recombinant DNA technology to form the pBR-M1-WT vector. A pBR-M1-c6 vector is constructed by a similar method, wherein M1-c6 is a M1 virus having a sequence shown as SEQ ID NO: 15, an amino acid residue at the 358th site of the NS3 protein of M1-c6 virus is M, and an amino acid residue at the 4th site of the E2 protein is E.
5. Full-length genome plasmids of mutant viruses: mutations are introduced into the pBR-M1-WT vectors by a site-directed mutation technology to form pBR-M1-NS3M (i.e. an M358L mutation of an amino acid residue at the 358th site of the comprised NS3 protein), pBR-M1-E2M (i.e. a K4N mutation of an amino acid residue at the 4th site of the E2 protein), and pBR-M1-N3E2M (i.e. an M358L mutation of an amino acid residue at the 358th site of the NS3 protein and a K4N mutation of an amino acid residue at the 4th site of the E2 protein); mutations are introduced into pBR-M1-c6 to form pBR-M1-c6v1 (i.e. an M358L mutation of an amino acid residue at the 358th site of the contained NS3 protein and an E4D mutation of the amino acid residue at the 4th site of the E2 protein).
6. DH5α competent cells, high-purity plasmid micro-extraction kits; JM110 competent cells; and Lipofectamine RNA iMAX Transfection Reagent.
7. RNA extraction: TRIzol; chloroform, isopropanol, and anhydrous ethanol.
8. Reverse transcription: Random hexamer, dNTP, MMLV, and RNaseOUT.
9. PCR: Q5 high-fidelity enzymes, and PCR primers shown in Table 3. The primers are synthesized by Invitrogen.
10. Agarose gel DNA recovery kits.
11. DNA electrophoresis: Agarose, SYBR green, and DNA marker.

TABLE 3
List of PCR primers

| Mutant | | |
|---|---|---|
| protein | Forward Primers (F) | Reverse Primers (R) |
| NS3 | GCTCCTCCTTTCCATTGC | TTCTTCTCTTTCTTGGTGCC |
| | (SEQ ID NO: 1) | (SEQ ID NO: 2) |
| E2 | TGATGATGTGCGTCTTAGC | ATCGTGCCTCGAATAGCG |
| | C (SEQ ID NO: 3) | (SEQ ID NO: 4) |

### Method:

### 1. Construction of Vectors for Full-Length Gene Plasmids of Viruses

pBR-M1-WT and pBR-M1-c6 bacteria were added into 5 mL of LB media and shaken at 37° C. overnight. Plasmids were extracted by using a plasmid micro-extraction kit, and the DNA concentration of the plasmids was measured by using Nanodrop. A PCR amplification system is shown in Table 4.

TABLE 4
PCR amplification system for site-directed mutation

| Reagent | Volume (μL) |
|---|---|
| 2× Phanta Max Buffer | 25 |
| dNTP Mix (10 mM each) | 1 |
| DNA | 1 |
| Forward primer | 1 |
| Reverse primer | 1 |
| Phanta Max Super-Fidelity | 1 |
| DNA Polymerase (1 U/μl) | |
| ddH₂O | 20 |

Reaction conditions: pre-degeneration was carried out at 98° C. for 3 min; amplification was carried out at 98° C. for 15 s, at 58° C. for 15 s, and at 72° C. for 45 s, respectively, and was cycled for 35 times; extension was carried out at 72° C. for 5 min, and after the reaction was completed, the system was cooled to 4° C. Whether products existed and the size was correct were determined by means of agarose gel electrophoresis.

Enzyme cleavage of vector plasmids: enzyme cleavage systems for NS3 and E2 mutations shown in Table 5 were prepared:

TABLE 5
Restrictive endonuclease enzyme cleavage system

| NS3 | E2 |
|---|---|
| pBR-M1-WT | pBR-M1-WT |
| 1 μg | 1 μg |
| 10× buffer 2 μL | 10× buffer 2 μL |
| SpeI 1 μL | apaI 1 μL |
| SwaI 1 μL | XhoI 1 μL |
| ddH₂O 6 μL | ddH₂O 6 μL |

The systems reacted at 37° C. for 1 h; plasmids after enzyme cleavage were recovered by using a kit, and the concentration was measured.

The vector and PCR fragments were assembled by using a Gibson Assembly Master Mix kit. Reaction system: PCR fragment 1+PCR fragment 2+plasmids+2×Mix+H₂O=1+1+1+10+7 (μL). Reaction conditions: 50° C., 1 h.

Transformation and cloning: 100 μL of DH5α competent cells were transformed with 10 μL of conjugates and were selected on an Ampicillin-resistant plate. Clones were selected for sample sequencing. A double-site mutant virus was obtained by an NS3 mutation on plasmids of the vector with an E2 mutation.

### 2. Preparation of Site-Directed Mutant Viruses

pBR-M1-NS3M, pBR-M1-E2M, pBR-M1-N3E2M, and pBR-M1-c6v1 bacteria were inoculated into 5 mL of LB media and shaken at 37° C. overnight. Plasmids were extracted by using a kit, and the concentration was measured. JM110 were transformed (the transformation efficiency of the JM110 competent cells was relatively low, a relative large amount of plasmids were required to transform 100 μL of competent cells, 300 μL of LB solution was added, and after being recovered for 1 h, the cells were coated on a plate) and selected on an Ampicillin-resistant plate. Monoclones were selected and added into 500 μL of LB/Ampicillin media, the bacteria were shaken at 37° C. for 12 h, the culture system was amplified, and the bacteria were shaken at 37° C. for 14 to 16 h. When the concentration of the bacterial solution was appropriate, the bacteria were stored with glycerol at the final concentration of 15 to 30%. Plasmids were extracted by using an endotoxin-free plasmid extraction kit, and the concentration was measured. An XbaI endonuclease for linearizing plasmids is shown in Table 6:

TABLE 6
Plasmid linearization enzyme cleavage system

| Reagent | Volume (μL) |
|---|---|
| 10× buffer | 10 |
| XbaI | 5 |
| DNA(10 μg) | x |
| ddH₂O | 85-x |

The system was divided into two tubes and reacted at 37° C. for 2 h. Proteinase K digestion: Proteinase K (20 mg/mL) was diluted 10 times, 2.5 μL of the diluted Proteinase K was added into each tube, 5 μL of 10% SDS was added, and incubated at 50° C. for 30 min. Linearized plasmids after enzyme cleavage were recovered by using a kit, and the DNA concentration was measured. In vitro transcription: the reaction was carried out at 37° C. for 2 h, after the reaction, products were not cryopreserved and used directly in the next operation. A system is shown in Table 7:

TABLE 7
In vitro transcription system

| Reagent | Sample volume (μL) |
|---|---|
| SP6 Transcription 5× Buffer | 4 |
| rNTPs (100 mM) | 1 + 1 + 1 + 0.6 |
| (A + C + U + G) | |
| Linearized DNA template | 8.4 |
| (1 to 2 μg) | |
| Ribo m⁷G Cap Analog, | 2 |
| 40 mM | |
| Enzyme Mix | 2 |
| Total volume | 20 |

DNA template removal: RQ1 RNase-Free DNase (1 U/μL) was added into the DNA template at 1 U/μg, and reacted at 37° C. for 15 min. RNA transfection: Vero cells were inoculated into a 6-well plate at 3×10⁵ cells/well one day in advance and cultured with 1.5 mL of complete media; 125 μL of Opti-MEM was uniformly mixed with 3.75 μL of LipomRNA, 125 μL of Opti-MEM was uniformly mixed with 2.5 μg of RNA respectively, then the two were placed into a tube and uniformly mixed, and stood at room temperature for 5 min; a complex of RNA and the transfection reagent was added into the cell culture dish, whether the cytopathic morphology appeared was observed in the next 2 to 4 days, a supernatant was collected, and the virus was amplified with Vero cells.

### 3. RNA Extraction by a TRIzol Method

Fully lysed the cells by pipetting with TRIzol. If precipitates appeared, the cell solution was centrifuged at 12,000 g and 4° C. for 10 min, and a supernatant was collected. 200 μL of chloroform was added, the mixture was shaken violently and uniformly mixed, and the mixture was stood at room temperature for 3 min. The mixture was centrifuged at 12,000 g and 4° C. for 15 min, about 500 μL of upper aqueous phase was sucked into a new EP tube. 500 μL of isopropanol was added, and the mixture was gently mixed by reversing and stood at room temperature for 10 min. The mixture was centrifuged at 12,000 g and 4° C. for 10 min, and a supernatant was removed. Precipitates were washed with 500 μL of pre-cooled 75% ethanol, the mixture was centrifuged at 12,000 g and 4° C. for 5 min, and a supernatant was removed. After being dried in air, RNA precipitates were dissolved in an appropriate amount of DEPC-treated water. The RNA concentration was measured by using Nanodrop.

### 4. Reverse Transcription

The reverse transcription was carried out by using MMLV reverse transcriptase:

TABLE 8

| | Volume (μL) |
|---|---|
| 50 μM random | 1 |
| hexamer | |
| 10 mM dNTP | 1 |
| RNA (1 to 5 μg) | x |
| DEPC-treated water | 10-x |

Various reaction components shown in Table 8 were added, and the system was pre-degenerated at 65° C. for 5 min and placed on ices immediately for 2 to 3 min;

4 μL of 5× buffer reaction solution, 2 μL of RNaseOUT regent, 1 μL of DTT, and 1 μL of reverse transcriptase MMLV were added, and the system reacted at 25° C. for 10 min, at 37° C. for 50 min, and at 70° C. for 15 min.

### 5. PCR

Reversely transcribed cDNA was diluted 2 to 5 times, and DNA was subjected to PCR amplification by using a Q5 high-fidelity enzyme (the genome of M1 was divided into 10 fragments and amplified):

TABLE 9

| | Volume (μL) |
|---|---|
| 2× Mix | 25 |
| 10 μM F | 1 |
| 10 μM R | 1 |
| cDNA | 1 |
| ddH₂O | 22 |

Reaction conditions: predegeneration was carried out at 98° C. for 30 s; amplification was carried out at 98° C. for 10 s, at 58° C. for 30 s, and at 72° C. for 1 min, and was cycled for 35 times; extension was carried out at 72° C. for 2 min, and after the reaction was completed, the system was cooled to 4° C.

Detection of PCR products by means of 1% agarose gel electrophoresis: 0.5 g of agarose gel was weighted and added into 50 mL of water, the mixture was heated to dissolve the agarose gel thoroughly, after the mixture was cooled to about 50° C., an SYBR green dye was added in a ratio of 1/10000, the mixture was poured into a gel preparation plate, a comb was inserted; after completely solidifying, the mixture was placed into an electrophoresis tank at 5 μL/well and subjected to electrophoresis at a voltage of 100 V for about 40 min; and DNA products were observed and photographed by using a Tanon imager.

TABLE 10

| | Volume (μL) |
|---|---|
| 5× buffer | 4 |
| Vector | 1 |
| Exnase | 2 |
| DNA(10 to 100 ng) | x |
| ddH₂O | 13-x |

6. The PCR Products were Delivered to Thermo Scientific for Sequencing.

### Results:

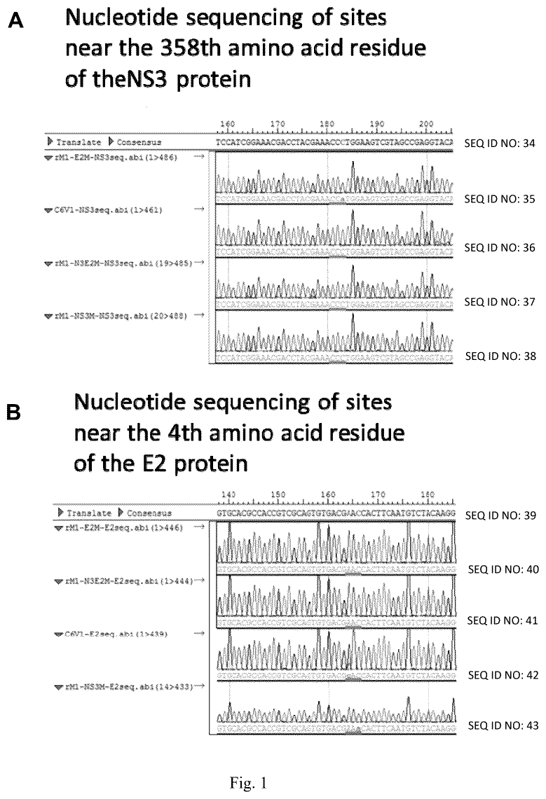

As shown in FIG. 1, by comparing gene sequences, it is found that all the site-directed mutant viruses are constructed successfully and are the following four mutant viruses, respectively:
(1) rM1-NS3M: an amino acid residue at the 358th site of the NS3 protein of a M1 virus having a sequence shown as SEQ ID NO: 5 mutates from methionine (M) into leucine (L);
(2) rM1-E2M: an amino acid residue at the 4th site of the E2 protein of a M1 virus having a sequence shown as SEQ ID NO: 5 mutates from lysine (K) into asparagine (N);
(3) rM1-N3E2M: an amino acid residue at the 358th site of the NS3 protein of a M1 virus having a sequence shown as SEQ ID NO: 5 mutates from methionine (M) into leucine (L); and an amino acid residue at the 4th site of the E2 protein of the M1 virus mutates from lysine (K) into asparagine (N); and
(4) rM1-c6v1: an amino acid residue at the 358th site of the NS3 protein of a M1 virus (i.e. M1-c6) having a sequence shown as SEQ ID NO: 15 mutates from methionine (M) into leucine (L), and an amino acid residue at the 4th site of the E2 protein mutates from glutamic acid (E) into aspartic acid (D).

### Conclusions:

4 mutant viruses rM1-NS3M, rM1-E2M, rM1-N3E2M, and rM1-c6v1 are constructed by means of site-directed mutation. The mutant viruses rM1-NS3M, rM1-E2M, and rM1-N3E2M are obtained on the basis of site-directed mutations of the M1 virus having the sequence shown as SEQ ID NO: 5; and rM1-c6v1 is obtained on the basis of site-directed mutations of the M1 virus (M1-c6) having the sequence shown as SEQ ID NO: 15.

### Example 2 Anti-Tumor Effects of Four Mutant M1 Viruses on a Variety of Tumor Cells

### 1. Experimental Materials and Instruments

### 1.1 Main Drugs, Reagents and Preparations

Main drugs and reagents:
M1 viruses: rM1-WT, rM1-NS3M, rM1-E2M, rM1-N3E2M, M1-c6, and M1-c6v1
Cell Counting Kit-8 (CCK-8) purchased from Dojindo China Co., Ltd. (item No.: CK-04)
Potassium chloride purchased from Merck Chemicals (Shanghai) Co., Ltd. (batch No.: K46837809603)
Potassium dihydrogen phosphate purchased from Merck Chemicals (Shanghai) Co., Ltd. (batch No.: AM1217539805)
Sodium bicarbonate purchased from Merck Chemicals (Shanghai) Co., Ltd. (batch No.: K49804023804)
Disodium hydrogen phosphate dodecahydrate purchased from Chengdu Huayi Pharmaceutical Excipient Manufacturing Co., Ltd. (batch No.: 20170402)
Anhydrous calcium chloride purchased from Hebei Huachen Pharmaceutical Co., Ltd. (batch No.: 171009)
Magnesium chloride hexahydrate purchased from Chengdu Huayi Pharmaceutical Excipient Manufacturing Co., Ltd. (batch No.: 20170807)
Mannitol purchased from France Roquette company (batch No.: E939X)·
Trehalose purchased from U.S. Pfanstiehl (batch No.: 36358A)
Human serum albumin purchased from Shenzhen Weiguang Biological Products Co., Ltd (batch No.: 20171144B)

### 1.2 Main Instruments

TABLE 11
Main experimental instruments

| Instrument | Manufacturer | Model |
|---|---|---|
| Inverted microscope | Nikon | ECLIPSE Ti-S |
| Biological safety | Thermo Fisher | 1374 |
| cabinet | (Suzhou) Instruments | |
| Cell counting and | Chemometec | Nucleocounter |
| analysis instrument | | NC200 |
| Electric-heated | Shanghai Yiheng | HWS-24 |
| thermostatic | Technology Instrument | |
| water bath | | |
| Desktop computer | Dell | OptiPlext 3020 |
| Refrigerator | Panasonic | BCD-Z51WZ |
| Cell incubator | Thermo Fisher | 3111 |
| Absorbance | Biotek | ELX800 |
| microplate reader | | |
| Clean bench | Suzhou Antai | SW-CJ-2FD |
| Centrifuge | Jintan Kexi | 80-2 |
| | Instruments | |

### 2. Sources of Experimental Cells

The cells were purchased from ATCC or National Collection of Authenticated Cell Cultures.

### 3. Experimental Methods

### Cell Culture

Appropriate culture conditions were selected according to instructions of various cells, and the cell amplification and passage was carried out according to passage ratios on the instructions.

### Experimental Grouping and Processing

Inhibitory effects of M1-c6v1 on the growth of various cells were detected. Specific grouping and processing are shown in Table 12.

TABLE 12
Grouping and processing of an inhibitory effect
experiment of M1-c6v1 on the growth of cells

| Group | Processing |
|---|---|
| Blank control | without cells + |
| | excipient solutions |
| Solvent control | excipient solutions |
| Positive control | 15 mg/mL or 20 mg/mL |
| | 5-Fu solution |
| Positive solvent | 1× PBS |
| control | |
| M1-c6v1 | M1-c6v1 at various |
| | concentrations |

After being subcultured to the logarithmic growth phase, the cells were digested, the cells were inoculated into a 48-well plate at a corresponding density, experiments were set according to the above groups, 3 duplicated wells were provided for each group, and the blank control group was not inoculated with the cells. After the cells were subjected to adherent culture for 24 h, the sample were added, the culture was continued for 72 h, and the cell viability was detected at the end point.

### Detection of the Cell Viability by a CCK-8 Method

The original culture solutions in the 48-well plate was removed, a color-developing agent (100% complete medium+10% CCK-8 solution) was slowly added along the wall at 200 μL/well, and after the cells were cultured at 37° C. for 0.5 to 3 h, absorbance values of various wells were detected in a wavelength of 450 nm by using an absorbance microplate reader.

### Calculation of Median Inhibitory Concentration (IC50) of the M1 Viruses Against Various Cells

Cell growth inhibition rates of various wells were calculated according to the detected absorbance values of various wells by a formula of cell growth inhibition rate (IR) (Average OD_{negative}-ODₑₓₚₑᵣᵢₘₑₙₜ)/(Average OD_{negative}-Average OD_{blank})×100%, cell growth inhibition curves were drawn by using GraphPad Prism 7.0 software, and a median inhibitory concentration (IC50) value of M1-c6v1 or rM1-c6 against tumor cells was calculated by a log (inhibitor) vs. response-variable slope (four parameters) analysis equation.

### Detection of the Cell Activity by Using MTT

The cells were inoculated into a 24-well plate at 20,000 cells/well, with 500 μl of medium per well, and the cells were subjected to adherent culture for 24 h. The wild-type and the mutant viruses were diluted in a 10-fold equal ratio, and the viruses were added into the 24-well plate in 10 MOI. After the cells were infected with the viruses for 72 h, 50 μL of MTT was added into each well and uniformly mixed with the cells, and the 24-well plate was placed in the incubator for 2 to 4 h. A supernatant was sucked away carefully, 500 μL of DMSO was added into each well to dissolve bluish violet crystalline formazan, which was thoroughly dissolved and mixed on a microporous oscillator, and the absorbance was detected in a wavelength of 570 nm by using the microplate reader. The experiment was repeated at least 3 times. Absorbance values of various groups were normalized according to the blank control group, a relative cell survival rate of each processing group was calculated, and a survival curve of the cells was drawn.

### Statistics Processing

All the experiments were repeated twice or more times separately, and experimental results were recorded as mean value±standard deviation.

### 4. Experimental Results

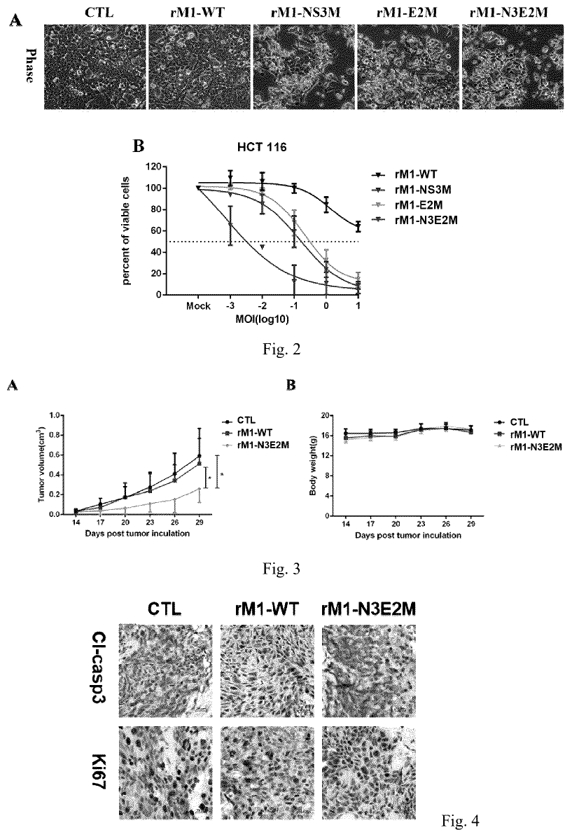

As shown in FIG. 2, part A, it is observed under a microscope that after being infected with the rM1-NS3M and rM1-E2M viruses, the infected cells have lesions, which indicates that a single-site mutation can independently enhance the replication and oncolytic effect of the M1 virus in tumor cells. Compared with the wild-type and the single-site mutant viruses, after the cells were infected with the rM1-N3E2M double-site mutant virus, an infection rate is more than 90%, and most of the cells have lesions. Kill effects of the rM1-WT, rM1-NS3M, rM1-E2M, and rM1-N3E2M on HCT 116 cells are further compared by an MTT method. Consistent with the observation under the microscope, dose-effect curves of the rM1-NS3M and rM1-E2M viruses shift to the left, the EC50 shift is respectively increased by 80 times and 60 times according to calculation, a dose-effect curve of the rM1-N3E2M virus shift to the left to a larger extent, and the EC50 shift is increased by 7,600 times (see FIG. 2, part B), which indicates that two mutant sites synergistically enhance the oncolytic effect of the virus.

Oncolytic effects and safety of rM1-WT and 3 M1 virus mutants, i.e. rM1-NS3M, rM1-E2M, and rM1-N3E2M, are compared by using more tumor cells and normal cells, and results are shown in Table 13 to Table 15. The results show that: compared with the rM1-WT virus, the 3 M1 virus mutants have improved oncolytic effects to different extents, and are not obviously toxic to the detected normal cells.

TABLE 13
Kill effect of rM1-NS3M to tumor cells
(denoted as a survival rate % of tumor cells)

| | | Survival | Survival |
|---|---|---|---|
| | | rate % of | rate % of |
| | | rM1-WT | rM1-NS3M |
| Cell line | Disease source | group | group |
| HCT-8 | Colorectal | 64.1 | 10.7 |
| | cancer | | |
| SW620 | Colorectal | 21.7 | 6.8 |
| | adenocarcinoma | | |
| SW480 | Colorectal | 55.3 | 8.1 |
| | cancer | | |
| HCT-15 | Colorectal | 29.3 | 19.8 |
| | adenocarcinoma | | |
| Huh-7 | Liver cancer | 30.9 | 10.8 |

TABLE 14
Kill effect of rM1-E2M to tumor cells
(denoted as a survival rate % of tumor cells)

| | | Survival | Survival |
|---|---|---|---|
| | | rate % of | rate % of |
| | | rM1-WT | rM1-E2M |
| Cell line | Disease source | group | group |
| HCT-8 | Colorectal | 64.1 | 9.4 |
| | cancer | | |
| SW620 | Colorectal | 21.7 | 6.9 |
| | adenocarcinoma | | |
| SW480 | Colorectal | 55.3 | 7.2 |
| | cancer | | |
| HCT-15 | Colorectal | 29.3 | 15.2 |
| | adenocarcinoma | | |
| Huh-7 | Liver cancer | 30.9 | 8.5 |

TABLE 15
Kill effect of rM1-N3E2M to tumor cells
(denoted as a survival rate % of tumor cells)

| | | Survival | Survival |
|---|---|---|---|
| | | rate % of | rate % of |
| | | rM1-WT | rM1-N3E2M |
| Cell line | Disease source | group | group |
| HCT-8 | Colorectal | 64.1 | 8.7 |
| | cancer | | |
| SW620 | Colorectal | 21.7 | 10.3 |
| | cancer | | |
| SW480 | Colorectal | 55.3 | 19.6 |
| | cancer | | |
| Huh-7 | Liver cancer | 30.9 | 6.9 |
| SK-HEP-1 | Liver cancer | 85.9 | 44.6 |
| MDA-MB-231 | Breast cancer | 17.0 | 3.0 |
| MDA-MB-435S | Breast cancer | 38.0 | 4.0 |
| BT-20 | Breast cancer | 20.0 | 8.0 |
| BT549 | Breast cancer | 98.0 | 65.0 |
| T47D | Breast cancer | 22.0 | 3.0 |
| ZR-75-1 | Breast cancer | 81.0 | 42.0 |
| SW1990 | Pancreatic | 32.9 | 16.1 |
| | cancer | | |
| MIA PACA-2 | Pancreatic | 45.2 | 27.7 |
| | cancer | | |
| BxPC-3 | Pancreatic | 23.9 | 10.1 |
| | cancer | | |
| ScaBER | Bladder cancer | 6.7 | 6.6 |
| SK-BR-3 | Breast cancer | 6.0 | 5.0 |
| MDA-MB-468 | Breast cancer | 7.0 | 2.0 |
| L-02 | Normal | 101.6 | 92.6 |
| | hepatocytes | | |
| NCM460 | Normal | 102.8 | 87.9 |
| | colorectal cells | | |

Similarly, the oncolytic effects and safety of the M1-c6 and M1-c6v1 viruses mutant are compared by using a variety of tumor cell lines and normal cells, and results are shown in Table 16. M1-c6v1 has a significant kill effect on a majority of malignant tumor cells. M1-c6v1 also has a significant kill effect on a majority of murine malignant tumor cells. Compared with the kill effect (IC50) of M1-c6, IC50 of M1-c6v1 is reduced by 1.3 to 20.5 times relative to IC50 of M1-c6, which indicates that the oncolytic effect of M1-c6v1 is generally better than that of M1-c6.

TABLE 16
Kill effects of M1-c6v1 and
rM1-c6 on malignant tumor cells

| | | | IC50 (MOI) | IC50 (MOI) |
|---|---|---|---|---|
| | | | of M1-c6v1 | of M1-c6 |
| | | | mean value ± | mean value ± |
| | | | standard | standard |
| No. | Tumor type | Cell name | deviation | deviation |
| 1 | Human liver | Hep 3B | <0.001 | <0.001 |
| 2 | cancer cell | Hep G2 | <0.001 | <0.001 |
| 3 | | PLC | <0.001 | <0.001 |
| 4 | | SNU-387 | 0.0103 ± 0.0063 | 0.0499 ± 0.0186 |
| 5 | | SNU-423 | 0.0479 ± 0.0150 | 0.0619 ± 0.0068 |
| 6 | | SNU-449 | 0.2054 ± 0.1898 | 0.3806 ± 0.4177 |
| 7 | | SNU-475 | <0.001 | 0.0041 ± 0.0031 |
| 9 | Human lung | SHP-77 | 0.0574 ± 0.0420 | 0.0861 ± 0.0607 |
| | cancer cell | | | |
| 10 | Human colon | HCT 116 | 0.0350 ± 0.0426 | 0.7181 ± 0.7789 |
| 11 | cancer cell | HCT-8 | 0.0174 ± 0.0062 | 0.0365 ± 0.0188 |
| 12 | Human | UM-UC-3 | 0.01450 ± 0.0233 | 0.0233 ± 0.0090 |
| | bladder | | | |
| | transitional | | | |
| | cell | | | |
| | carcinoma | | | |
| 13 | Osteosarcoma | MNNG/ | <0.001 | <0.001 |
| | cell | HOSCl #5 | | |
| 15 | Human | C-33 A | 4.77 × 10⁻⁶ ± | 3.61 × 10⁻⁶ ± |
| | cervical | | 4.52 × 10⁻⁷ | 2.19 × 10⁻⁶ |
| | cancer cells | | | |
| 16 | Human | A-375 | 0.1360 ± 0.0713 | 0.7155 ± 0.6710 |
| | malignant | | | |
| | melanoma | | | |
| | cell | | | |
| 17 | Human | U87MG | 0.0007 ± 0.0014 | 0.0003 ± 0.0006 |
| | glioma cells | | | |
| 18 | Murine liver | Hepa 1-6 | 0.0202 ± 0.0081 | 0.0181 ± 0.0077 |
| | cancer cell | | | |
| 19 | Murine breast | 4T1 | 0.7666 ± 2.2841 | 1.1892 ± 1.7708 |
| | cancer cell | | | |
| 21 | Murine colon | MC38 | <0.001 | <0.001 |
| 22 | cancer cell | C26 | 0.0089 ± +0.0086 | 0.0257 ± 0.038 |
| 23 | Murine | B16-F10 | <0.01 | <0.01 |
| | cutaneous | | | |
| | melanoma | | | |
| | cell | | | |

### 5. Experimental Conclusions

The mutant strains rM1-NS3M, rM1-E2M, rM1-N3E2M, and M1-c6v1 have significant kill effects on a majority of malignant tumor cells of the human and murine malignant tumors.

### Example 3 Study on the In Vivo Efficacy and Safety of the rM1-N3E2M Virus

### Materials:

1. rM1-N3E2M and rM1-WT viruses
2. Colorectal cancer cell lines HCT 116
3. 30 female BALB/c-nu/nu nude mice at the age of 6 to 8 weeks

### Method:

1. Female nude mice at the age of 4 to 6 weeks were purchased, sufficient HCT 116 cells were cultured in advance, digested cells were resuspended with aseptic PBS, counted, and prepared into cell suspensions as needed, and the HCT 116 cells were subcutaneously injected into backs of the nude mice at 5×10⁶ cells/100 μL. After tumors grew to about 50 mm³, the nude mice were grouped randomly, and administration was carried out for 6 consecutive days. Lengths and widths of the tumor were measured every three days, volumes (volume=(length×width²)/2) of the tumors were calculated, and growth curves were drawn. The tumor tissues and normal organs such as liver, heart, brain, and lung were isolated on the third day after administration, and fixed with 4% paraformaldehyde for immunohistochemical experiments.

2. Immunohistochemical experiment: the tumor tissues and the normal organs of the nude mice were selected and fixed with 4% paraformaldehyde, and the samples were delivered to Guge Biotechnology for detection of the quantity of Cleaved-caspase 3 and Ki67.

### Results:

1. Subcutaneous tumor-bearing models were constructed on the BALB/c nude mice, and the administration (intravenous injection) was carried out for 6 consecutive times, survival states of the nude mice were observed. It is found that compared with the control group, weights of the nude mice of the administration groups do not change significantly (see FIG. 3, part B), and mental states are good, which indicates that the rM1-N3E2M mutant virus has good safety to a certain extent. Compared with the control group, the volumes of the tumors of the nude mice of the rM1-N3E2M administration groups are reduced significantly (see FIG. 3, part A), which indicates that the rM1-N3E2M virus can reach tumor cells and kill the tumor cells to inhibit the growth of the tumor.

2. In order to study an inhibitory effect of rM1-N3E2M on the growth of a tumor in vivo, after the administration was carried out for 3 days, the tumor tissue was dissected, and cleaved caspase-3 (Cl-casp3) and Ki67 were subjected to immunohistochemical staining. Cl-casp3 is a marker for apoptosis, and its expression quantity indicates the degree of apoptosis and necrosis of tumor cells. Ki67 is a marker for proliferation, and its expression quantity indicates the proliferation ability of tumor cells. As shown in FIG. 4, the brown color refers to positive signals. The rM1-N3E2M virus can significantly induce up-regulation of the expression of Cl-casp3 and down-regulation of the expression of Ki67, which indicates that it can induce apoptosis of tumor cells and a malignant phenotype of rapid proliferation of the tumor cells at the same time.

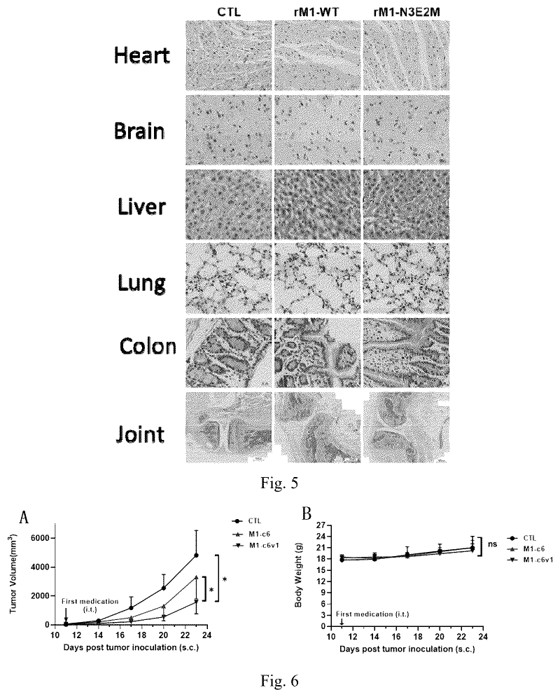

3. The safety of an oncolytic virotherapy is very important, it is proved that the rM1-N3E2M virus is safe to normal cell lines, but its safety in vivo needs to be further studied. Main organs including heart, brain, liver, lung, colon, arthrosis, and other tissues of the experimental animals of various groups were isolated on the third day after the nude mouse subcutaneous tumor-bearing models were administrated, and whether the expression of the rM1-N3E2M virus protein existed was observed by an immunohistochemical method. The rM1-N3E2M virus was obtained by continuous passage on a colorectal cancer cell line, and therefore, whether the virus replicated in colonic epithelial cells of the mice in addition to important organs such as heart and brain was also concerned. In addition, it is reported that a variety of alphaviruses can cause arthritis, and therefore, whether the two experimental viruses replicated in joint tissues are observed. It can be seen from FIG. 5 that the viruses do not replicate, the cell morphologies of various organs do not change, which proves that the rM1-N3E2M virus has good safety in the immunodeficient nude mice

### Conclusions:

The animal experiments verify that the rM1-N3E2M virus can effectively play a role in resisting a tumor in vivo, and inhibit the growth of the tumor; rM1-N3E2M induces apoptosis of a tumor tissue and inhibits a malignant phenotype of the tumor cells; during the experiments, the weights of the nude mice do not change significantly, virus replication is not detected in the normal tissues and organs, and therefore, the safety of rM1-N3E2M is good.

### Example 4 Effective Inhibition of the M1-c6v1 Virus Against a Tumor In Vivo

### Materials:

1. M1-c6v1 and M1-c6 viruses
2. Murine melanoma B16-F10 cells
3. 40 female C57 BL/6 mice at the age of 5 to 6 weeks

Method: female C57 BL/6 mice at the age of 5 to 6 weeks were purchased, sufficient B16-F10 cells were cultured in advance, digested cells were resuspended with aseptic PBS, counted, and prepared into cell suspensions as needed, and the B16-F10 cells were subcutaneously injected into backs of the mice at 5×10⁴ cells/100 μL. After tumors grew to about 60 mm³, the mice were grouped randomly, and intratumoral injection was carried out for 7 consecutive days. Lengths and widths of the tumors were measured every three days, volumes (volume=(length×width²)/2) of the tumors were calculated, and growth curves were drawn.

Results: subcutaneous tumor-bearing models were constructed on the C57 BL/6 mice, and after the intratumoral injection was carried out for 7 consecutive days, survival states of the mice were observed. It is found that compared with the control group, weights of the mice of the administration groups do not change significantly (see FIG. 6, part B), and mental states are good, which indicates that the M1-c6v1 mutant virus has good safety to a certain extent. Compared with the control group and the M1-c6 administration group, the volumes of the tumors of the mice of the M1-c6v1 administration groups are significantly reduced (see FIG. 6, part A), which indicates that the M1-c6v1 virus can kill the tumor cells to inhibit the growth of the tumor.

### Conclusions:

The animal experiments verify that the M1-c6v1 virus can effectively play a role in resisting a tumor in vivo, and inhibit the growth of the tumor; during the experiments, the weights of the mice do not change significantly, which indicates that the safety of M1-c6v1 is good.

### Example 5 Construction, Identification, and Efficacy Tests of M1 Site-Directed Mutant Virus Strains

### Process of Point Mutation:

A series of site-directed mutations were performed on the basis of rM1-WT viruses having a genome sequence shown as SEQ ID NO: 5 (by GenScript Biotech Co., Ltd.), and pBR-M1-E2-4K, pBR-M1-E2-4L, pBR-M1-E2-4I, pBR-M1-E2-4V, pBR-M1-E2-4S, pBR-M1-E2-4C, pBR-M1-E2-4L, and pBR-M1-E2-4D plasmids; and pBR-M1-NS3-358M, pBR-M1-NS3-358G, pBR-M1-NS3-358A, pBR-M1-NS3-358L, pBR-M1-NS3-358I, pBR-M1-NS3-358V, pBR-M1-NS3-358P, pBR-M1-NS3-358S, pBR-M1-NS3-358Q, pBR-M1-NS3-358T, pBR-M1-NS3-358C, pBR-M1-NS3-358N, pBR-M1-NS3-358F, pBR-M1-NS3-358Y, pBR-M1-NS3-358D, pBR-M1-NS3-358K, pBR-M1-NS3-358R, and pBR-M1-NS3-358H were respectively obtained.

### 5.1 Reagents and Consumables

### Main Drugs and Reagents

DMEM/F12 (Gibco, 11320-033)
MEM (Gibco, C11095500BT)
DMEM (Corning, 10-013-CVRC)
New-born calf serum (Hangzhou Tianhang Biotechnology, 22011-8615)
Fetal calf serum (Corning, 35-081-CV)
Protease K (Tiangen, RT403)
Trypsin (Thermo, 25200-072)
Lipofectamine Messenger MAX Transfection Reagent (Thermo, LMRNA003)
Opti-MEM I Reduced Serum Medium (1x) (Thermo, 31985-070)
Restriction endonuclease XbaI (NEB, R0145S)
Ribo m⁷G Cap Analog (Promega, P1711)
DNA purification and recovery kit (Tiangen, DP209)
PBS (Gibco, 20012027)
RiboMAX™ Large Scale RNA Production System (Promega, P1280)
Goscript™ Reverse Transcription System (Promega, A5001)
Total RNA extraction kit (Promega, LS1040)
Virus Production Serum Free Medium (VP-SFM) (Gibco, 11681-020)
GlutaMax I (100×) (Gibco, 35050061)
MEM NEAA (100×) (Gibco, 11140050)
Primers for E2 sequencing (GENEWIZ, order No. 80-423889537)
Primers for N3 sequencing (GENEWIZ, order No. 80-429401706 and 80-426066909)

### Preparation of Main Solutions

Preparation of a VP-SFM solution

10 mL of 100×MEM NEAA and 20 mL of 100× GlutaMax I were added into 1 L of VP-SFM, and the mixture was uniformly mixed and stored at 4° C. for later use.

### 5.2 Instruments and Equipment

TABLE 17
Main experimental instruments

| Instrument | Manufacturer | Model |
|---|---|---|
| Inverted microscope | Nikon | Eclipse TS2R |
| Biological safety cabinet | Thermo | 1374 |
| Electric-heated | Shanghai Yiheng | HWS-24 |
| thermostatic water bath | Technology Instrument | |
| Desktop computer | Dell | OptiPlext 3020 |
| Refrigerator | Panasonic | BCD-Z51WZ |
| Cell incubator | Thermo | 3111 |
| Absorbance microplate | Biotek | ELX800 |
| reader | | |
| Clean bench | Suzhou Antai | SW-CJ-2FD |
| Centrifuge | Jintai Kexi Instruments | 80-2 |
| Benchtop refrigerated | Eppendorf | 5424R |
| centrifuge | | |
| Cell counter | Nexcelom | Auto T4 |
| Low-speed benchtop | Shanghai Anting | TDL-80-2B |
| centrifuge | | |
| Thermal cycler | ABI | Simpliamp |

### 5.3 Experimental Cells

TABLE 18
Sources of experimental cells

| | | | Cell source | |
|---|---|---|---|---|
| | | | (bank level, | |
| No. | Tumor type | Cell name | record No.) | Medium |
| 1 | African green | Vero | Production | 3% NBS |
| | monkey | | cell bank, | DMEM/F12 |
| | kidney cell | | 3020014-01 | |
| 2 | Human colon | HCT-116 | Master cell | 10% FBS |
| | cancer cell | | bank, master | DMEM |
| | | | 20170505 | |
| 3 | Hamster | BHK-21 | Working cell | 10% FBS |
| | kidney cell | | bank, | MEM |
| | | | 20170331-071/100 | |

### 5.4. Test Procedure

### 5.4.1 Construction of Original Seed Lot Viruses

### (1) Linearization of Plasmids

TABLE 19
Plasmid linearization system

| | Addition amount |
|---|---|
| Components | (μL) |
| 10× Fast | 10 |
| Digest Buffer | |
| XbaI | 10 |
| Plasmids | 80 |
| (10 μg) + water | |

A. the system was prepared according to Table 19 in a PCR tube, and the tube was placed in a PCR instrument for incubation at 37° C. for 1 h; and
B. 5 μL of Protease K was added into each tube, and the tube was placed in the PCR instrument for incubation at 50° C. for 30 min.

### (2) Purification and Recovery of DNA

A. 500 μL of BL was added into an adsorption column and centrifuged at 12,000 rpm for 1 min;
B. linearized plasmids were transferred into a 1.5 mL EP tube, 500 μL of PB was added, and the mixture was uniformly mixed;
C. the mixture was transferred into the adsorption column, stood at room temperature for 2 min, and centrifuged at 12,000 rpm for 1 min, and a filtrate was removed;
D. 600 μL of PW was added, the mixture was stood for 2 min and centrifuged at 12,000 rpm for 1 min, and a filtrate was removed;
E. step D was repeated;
F. a filtrate was removed, the empty tube was centrifuged at 12,000 rpm for 2 min, opened lid, and dried at room temperature for 5 min;
G. 30 μL of aseptic DEPC-treated water was added, the mixture was stood for 1 min and eluted twice, and a filtrate was collected; and
H. DNA was quantified.

### (3) In Vitro Transcription

TABLE 20
Preparation of in vitro transcription system

| | Addition amount |
|---|---|
| Reagent | (μL) |
| SP6 Transcription | 8 |
| 5× Buffer | |
| rNTPs (100 mM) | 2 + 2 + 2 + 1.2 |
| (A + C + U + G) | |
| Linearized DNA template | 16.8 |
| (1 to 2 μg) + water | |
| Ribo m⁷G Cap | 4 |
| Analog, 40 mM | |
| Enzyme Mix | 4 |
| Total volume | 40 |

A. the system was prepared according to Table 20 in a PCR tube, and the tube was placed in a PCR instrument for incubation at 37° C. for 2 h; and
B. RQ1 RNase-Free DNase (1 μL/1 μg DNA) was added, and the tube was placed in the PCR instrument for incubation at 37° C. for 15 min.

### (4) RNA Transfection

### Planking of Cells

Appropriate culture conditions were selected according to instructions of various cells, and the cell amplification and passage was carried out according to passage ratios on the instructions. After being subcultured to the logarithmic growth phase, the cells were digested, and the Vero cells were inoculated into T25 culture flasks at a density of 4×10⁵/flask, placed in the cell incubator, and subjected to adherent culture for 24 hours.

### B. Transfection

250 μL of Opti-MEM medium and 7.5 μL of MessengerMAX were added into a 1.5 mL centrifuge tube, 125 μL of Opti-MEM medium and all the RNA were added into another centrifuge tube, the solutions in the two tubes were uniformly mixed and dropwise added to the Vero cells, and the cells were placed in the virus incubator for culture.

### (5) Collection of Viruses

The cell morphology and whether fluorescence appeared were observed every day, the cells were photographed for record when fluorescence was increased significantly, and when more than 90% of cells floated up, a Vero cell supernatant was collected and centrifuged at 4,000 rpm for 5 min. The supernatant was subpackaged into cryopreservation tubes, used as original seed lots, and stored with liquid nitrogen

### 5.4.2 Sequencing of Seed Lot Viruses

### (1) Extraction of RNA

A. the seed lot virus sample stored with liquid nitrogen was taken out and defrosted;
B. 100 μL of sample was sucked and placed into a 1.5 mL centrifuge tube;
C. 300 μL of lysate was added, and the mixture was eddied, centrifuged transitorily, and stood at room temperature for 5 min;
D. 300 μL of diluent was added, and the mixture was eddied, centrifuged transitorily, and stood at room temperature for 5 min;
E. 350 μL of anhydrous ethanol was added, and the mixture was eddied, centrifuged transitorily, and stood at room temperature for 5 min;
F. the mixture was transferred into a centrifuge column (provided in a kit), and was loaded and subjected to column chromatography in two times. The mixture was centrifuged at 12,000 rpm for 1 min, and a filtrate was removed;
G. 600 μL of RNA washing solution was added, the mixture was centrifuged at 12,000 rpm for 1 min, and a filtrate was removed; and
H. 50 μL of DNase I incubation solution shown as Table 21 was added to the center of an adsorption film and was stood at room temperature for 15 min;

TABLE 21
Preparation of DNase I incubation solution

| | Volume/ |
|---|---|
| Reagent | μL |
| 10× DNase I buffer | 5 |
| DNase I | 5 |
| Nuclease-free water | 40 |

I. 600 μL of RNA washing solution was added, the mixture was centrifuged at 12,000 rpm for 1 min, a filtrate was removed, the mixture was washed twice, and a filtrate was removed. The centrifuge column was placed on a collection tube again and centrifuged at 12,000 rpm for 2 min; and
J. the centrifuge column was transferred to an elution tube, 50 μL of nuclease-free water was added to the center of a film of the centrifuge column, stood at room temperature for 2 min and centrifuged at 12,000 rpm for 1 min, and RNA was stored at −80° C.
   (2) Reverse transcription, as Table 22:

TABLE 22
Reverse transcription RT-PCR two-step reaction system

| Components | Volume (μL) | Note |
|---|---|---|
| Template RNA | 10 | First step: |
| Oligo | 1.5 | 1. 70° C., 5 min |
| | | 2. place on an ice box for |
| | | fast cooling for 5 min |
| | | 3. Transitory centrifuge |
| 5× reaction buffer | 4.0 | Second step: |
| MgCl₂ | 2.0 | 4. uniformly mix, and add |
| dNTP Mix | 1.0 | 17 μL of reaction mixture |
| (10 mM) | | into each sample |
| Ribonuclease | 0.5 | 5. react at 25° C. for 5 min, |
| Inhibitor | | at 42° C. for 1 h, and at 70° C. |
| RTase | 1.0 | for 5 min, and store at 4° C. |

### (3) PCR Amplification

A. cDNA obtained at the previous step was used as a template for PCR of fragments. A reaction system is shown in Table 23:

TABLE 23
PCR reaction system

| | Volume |
|---|---|
| Components | (μL) |
| Q5 high-fidelity | 25 |
| 2× Master mix | |
| cDNA template | 1.0 |
| H₂O | 20 |
| Forward/reverse | 4 |
| primer pair | |
| (10 μM each) | |

B. reaction conditions are shown in Table 24:

TABLE 24
PCR reaction conditions

| | | Number |
|---|---|---|
| Temperature | Time | of cycles |
| 98° C. | 2 min | 1 cycle |
| 98° C. | 10 s | |
| 60° C. | 20 s | 30 cycles |
| 72° C. | 1 min | |
| 72° C. | 10 min | 1 cycle |

### (4) Sequencing

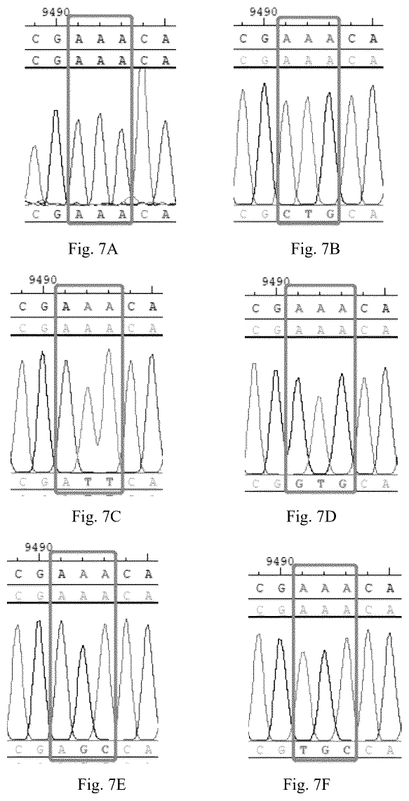

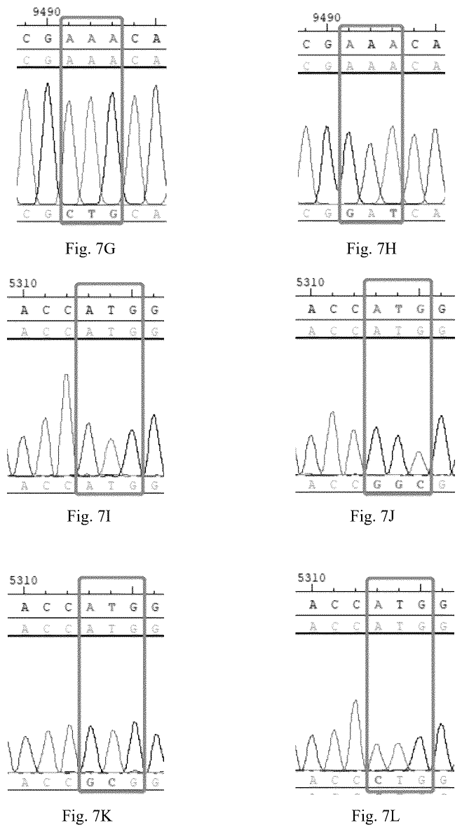

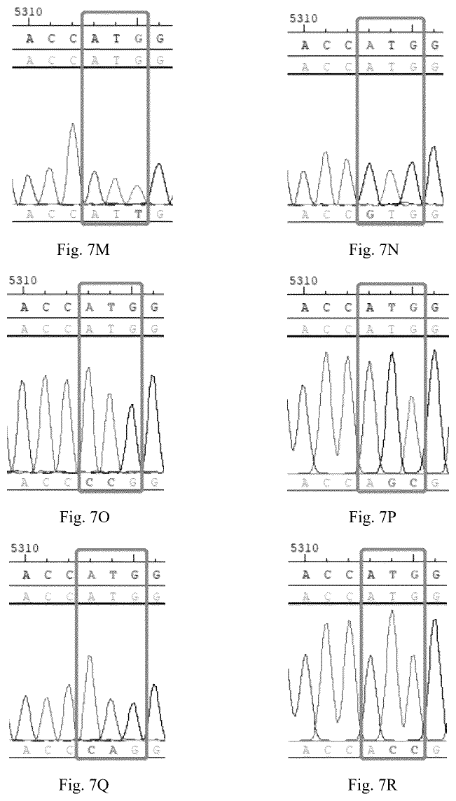

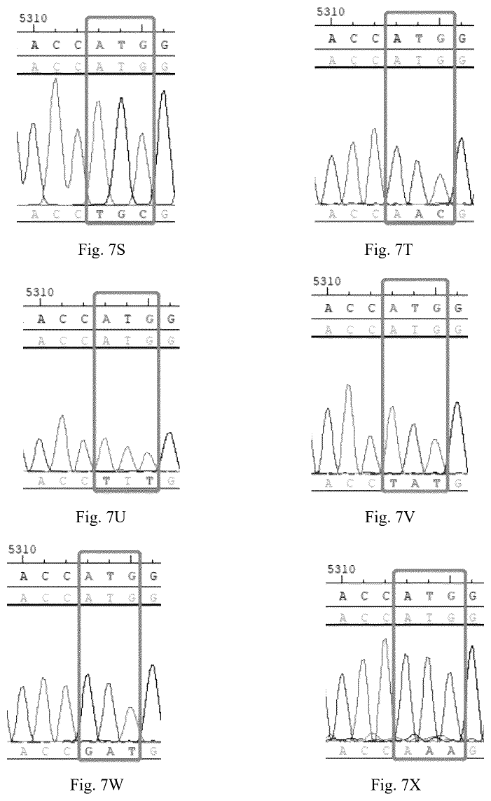

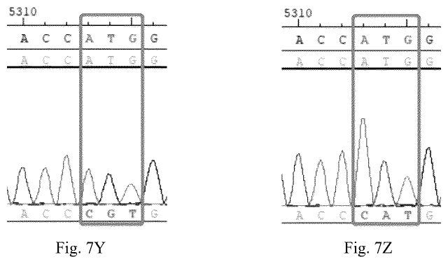

Sequencing results are shown in FIGS. 7A to 7Z. Various mutant viruses achieved site-directed mutation as expected.

### 5.4.3 Establishment of a Virus Working Bank

### (1) Planking of Cells, Inoculation of Viruses, and Collection of Samples

Appropriate cultural conditions were selected according to instructions of various cells, and the cell amplification and passage was carried out according to passage ratios on the instructions. After being subcultured to the logarithmic growth phase, the cells were digested, inoculated into T25 culture flasks at 6×10⁵ cells/flask, and subjected to adherent culture for 24 h;

After the cells were subjected to adherent culture for 24 h, the original medium was sucked away, a VP-SFM virus culture medium mixed with 20 μL of seed lot viruses was added, and placed in the virus incubator to wait for the cells to produce viruses. The cell morphology and whether fluorescence appeared were observed every 24 h, when more than 90% of cells floated up, the cells were photographed for record, and a Vero cell supernatant was collected and centrifuged at 4,000 rpm for 5 min. 100 μL of supernatant was placed into a 1.5 mL centrifuge tube and used as a sample for virus titer detection, and the rest was subpackaged into cryopreservation tubes (about 1.5 mL/tube) and stored with liquid nitrogen.

### (2) Virus Titer Detection

((1)) BHK-21 cells were planked in a 96-well plate at 1,500 cells/well, and cultured at 37° C. under a 5% CO₂ condition. After being planked, the cells were used within 12 to 36 hours.

((2)) an appropriate amount of MEM basic medium was poured into a loading slot, the medium was added into a 96-well plate at 180 μL/well and used as diluent for later use. The MEM diluents were prepared according to the number of samples to be detected, 2 duplications were prepared for each sample, and 4 duplications were prepared for a reference sample;

((3)) the samples to be detected were oscillated thoroughly in a vortex oscillator for 15 to 30 seconds. 20 μL of sample to be detected was added into wells of the 1st column of “((2))”. The mixture was uniformly mixed, and 20 μL of the mixture was sucked into wells of the next column. The sample was gradually diluted until wells of the 8th column. The tip should be changed for operation of each column, the diluted sample to be detected was fixed on a microplate mixer and oscillated at 3,000 rpm for 3 minutes;

((4)) the diluted virus solution was added to the cells at 20 μL/well, 10⁻³ to 10⁻⁸ virus solutions were added into the cells in the 96-well plate, and each plate was used for detection of two samples;

((5)) after the samples were loaded, the cells were cultured at 37° C. under a 5% CO₂ condition for 5 days (taking the day of virus loading as the first day);

((6)) 5 days later, whether the cells had an cytopathic effect (CPE) was observed under the microscope, and recorded; and

((7)) CCID50 values were calculated by a Spearman-Karber method. 1 g (CCID50×20/1000)=L−D (S-0.5), where L represents a logarithm of the maximum dilution, −1; D represents a difference between logarithms of the dilution, 1; and S represents a sum of proportions of positive wells (sum of the number of CPE/8)

Titer detection results of the working lot viruses are shown in Table 25.

TABLE 25
Titer results of working lot viruses

| | | Virus titer |
|---|---|---|
| | | CCID50/ |
| No. | Virus strain | mL |
| 1 | E2-4K | 2.00 × 10⁷ |
| 2 | E2-4L | 2.67 × 10⁶ |
| 3 | E2-4I | 1.84 × 10⁶ |
| 4 | E2-4V | 2.00 × 10⁶ |
| 5 | E2-4S | 5.83 × 10⁶ |
| 6 | E2-4C | 2.00 × 10⁶ |
| 7 | E2-4M | 5.21 × 10⁵ |
| 8 | E2-4D | 5.21 × 10⁷ |
| 9 | NS3-358M | 2.00 × 10⁷ |
| 10 | NS3-358G | 2.93 × 10⁷ |
| 11 | NS3-358A | 5.00 × 10⁶ |
| 12 | NS3-358L | 5.21 × 10⁷ |
| 13 | NS3-358I | 3.28 × 10⁷ |
| 14 | NS3-358V | 1.85 × 10⁷ |
| 15 | NS3-358P | 2.11 × 10⁷ |
| 16 | NS3-358S | 2.93 × 10⁵ |
| 17 | NS3-358Q | 2.20 × 10⁷ |
| 18 | NS3-358T | 3.75 × 10⁵ |
| 19 | NS3-358C | 2.46 × 10⁵ |
| 20 | NS3-358N | 9.26 × 10⁶ |
| 21 | NS3-358F | 8.89 × 10⁶ |
| 22 | NS3-358Y | 2.11 × 10⁶ |
| 23 | NS3-358D | 6.67 × 10⁶ |
| 24 | NS3-358K | 7.78 × 10⁷ |
| 25 | NS3-358R | 5.83 × 10⁶ |
| 26 | NS3-358H | 8.89 × 10⁶ |

### 5.4.4 Kill Effects of the Viruses on HCT 116 Cells

### (1) Inoculation of Cells and Administration

The cell amplification and passage was carried out according to culture conditions and passage ratios on instructions of various cells. After being cultured to the logarithmic growth phase, the cell were digested, and inoculated into a 48-well plate at a density of 10,000 cells/well, and a total volume of each well was 200 μL; blank control groups, negative control groups, and multiple gradients of the viruses: ((1)) 0.1 ((2)) 1 ((3)) 10 MOI experimental groups (3 duplicated plated for each group, see Table 26, A to H in the table represented different viruses) were set, the blank control groups were not inoculated with cells, and the negative control groups were inoculated with VP-SEM in the quantity equal to the inoculation dosage of the viruses.

TABLE 26
Administration design for 48-well plate

| | | | | | | | |
|---|---|---|---|---|---|---|---|
| Blank | Negative | Negative | Negative | Negative | Negative | Negative | Blank |
| control | control | control | control | control | control | control | control |
| Blank | A((1)) | A((2)) | A((3)) | B((1)) | B((2)) | B((3)) | Blank |
| control | | | | | | | control |
| Blank | C((1)) | C((2)) | C((3)) | D((1)) | D((2)) | D((3)) | Blank |
| control | | | | | | | control |
| Blank | E((1)) | E((2)) | E((3)) | F((1)) | F((2)) | F((3)) | Blank |
| control | | | | | | | control |
| Blank | G((1)) | G((2)) | G((3)) | H((1)_) | H((2)) | H((3)) | Blank |
| control | | | | | | | control |
| Blank | Negative | Negative | Negative | Negative | Negative | Negative | Blank |
| control | control | control | control | control | control | control | control |

After the viruses were loaded, the cells were continuously cultured for 72 h, the cell morphology was observed under the microscope at the end point and photographed for record, the cell viability was detected, and IC50 of various viruses on the detected cells were calculated.

### (2) Detection of the Cell Viability by a CCK-8 Method

The original culture solution in the 48-well plate was removed, a color-developing agent (90% complete medium+10% CCK-8 solution) was slowly added along the wall at 200 μL/well, and after the cells were cultured at 37° C. for 0.5 to 3 h, absorbance values of various wells were detected in a wavelength of 450 nm by using the absorbance microplate reader.

### (3) Calculation of Median Inhibitory Concentrations (IC50) of the Viruses Against Various Cells

Cell growth inhibition rates of various wells were calculated according to the detected absorbance values of various wells by a formula of cell growth inhibition rate (IR)=(Average ODₛₒₗᵥₑₙₜ-ODₑₓₚₑᵣᵢₘₑₙₜ)/(Average ODₛₒₗᵥₑₙₜ-Average OD_{blank})×100%, cell growth inhibition curves were drawn by using GraphPad Prism 8.0 software, and median inhibitory concentration (IC50) values of various viruses against tumor cells were calculated by a log (inhibitor) vs. response-variable slope (four parameters) analysis equation.

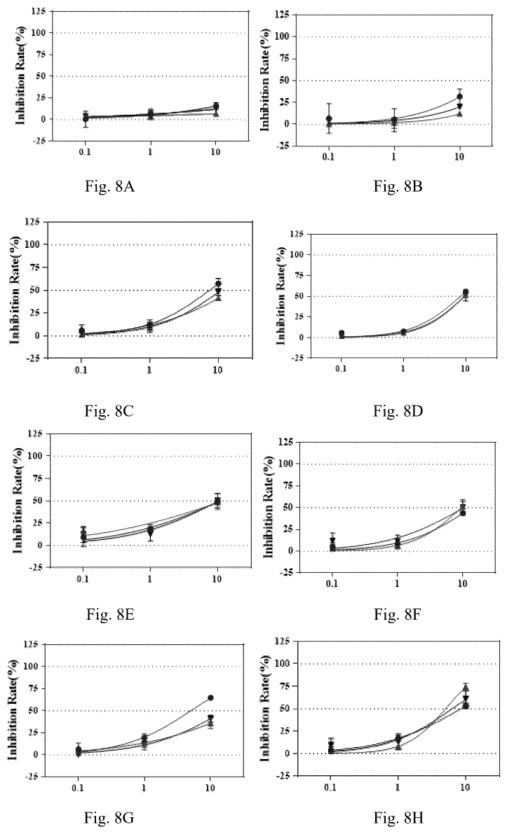

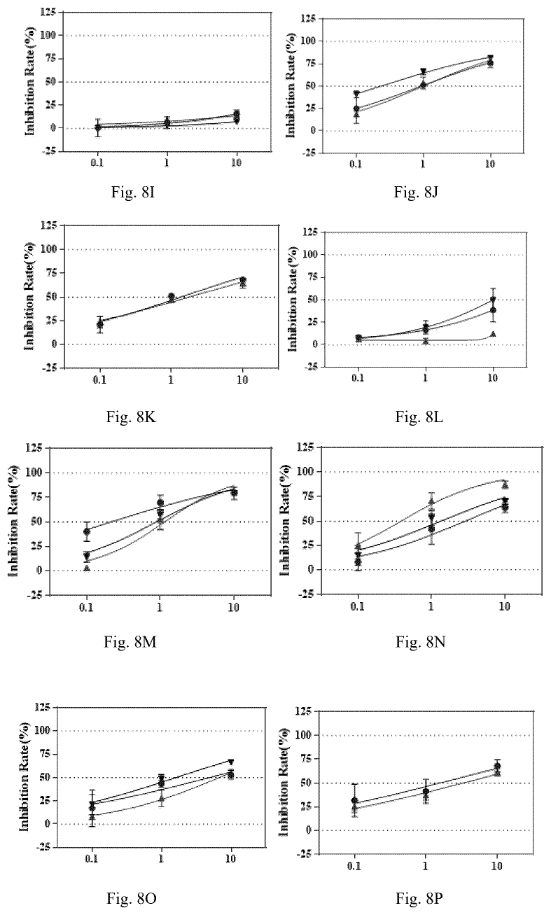

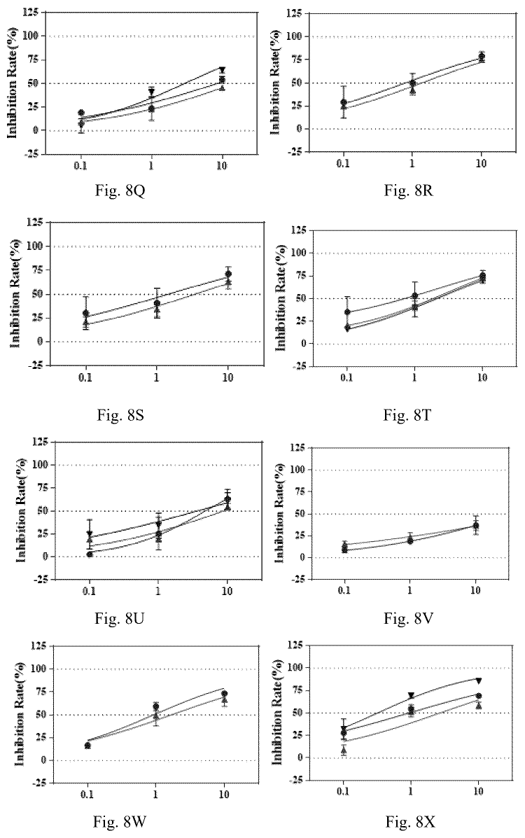

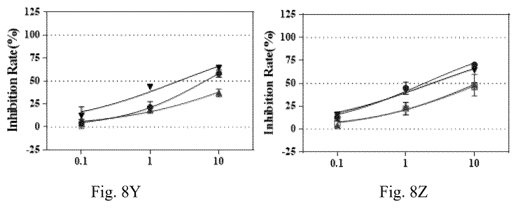

Kill curves of various site-directed mutant strains of the M1 virus are shown in FIGS. 8A to 8Z (note: each curve in the figures represents one independent experiment, the x-axis represents virus infection MOI, and the y-axis represents the inhibitory rate).

### REFERENCES

1. Greenman C, Stephens P, Smith R, et al. Patterns of somatic mutation in human cancer genomes. Nature 446:153-158, 2007.
2. Wood L D, Parsons D W, Jones S, et al. The genomic landscapes of human breast and colorectal cancers. Science 318:1108-1113, 2007.
3. Pavet V, Portal M M, Moulin J C, Herbrecht R, and Gronemeyer H. Towards novel paradigms for cancer therapy. Oncogene 30:1-20, 2011.
4. Workenhe S T and Mossman K L. Oncolytic virotherapy and immunogenic cancer cell death: sharpening the sword for improved cancer treatment strategies. Mol Ther 22:251-256, 2014.
5. Sun Q1, Barz M2, De Geest BG3, et al. Nanomedicine and macroscale materials in immuno-oncology. Chemical Society 2018 Nov. 22.
6. Tran E1, Robbins P F1, Rosenberg S A. ‘Final common pathway’ of human cancer immunotherapy: targeting random somatic mutations. Nature Immunology 2017 Feb. 15; 18(3): 255-262.
7. Das T, Jaffar-Bandjee M C, Hoarau J J, et al. Chikungunya fever: CNS infection and pathologies of a re-emerging arbovirus. Prog Neurobiol 91:121-129, 2010.
8. Kelvin A A. Outbreak of Chikungunya in the Republic of Congo and the global picture. J Infect Dev Ctries 5:441-444, 2011.
9. Moran T P, Burgents J E, Long B, et al. Alphaviral vector-transduced dendritic cells are successful therapeutic vaccines against neu-overexpressing tumors in wild-type mice. Vaccine 25:6604-6612, 2007.
10. Weaver S C, Salas R, Rico-Hesse R, et al. Re-emergence of epidemic Venezuelan equine encephalomyelitis in South America. VEE Study Group. Lancet 348:436-440, 1996.
11. Li X D, Qiu F X, Yang H, Rao Y N, and Calisher C H. Isolation of Getah virus from mosquitos collected on Hainan Island, China, and results of a serosurvey. Southeast Asian J Trop Med Public Health 23:730-734, 1992.
12. Wen J S, Zhao W Z, Liu J W, et al. Genomic analysis of a Chinese isolate of Getah-like virus and its phylogenetic relationship with other Alphaviruses. Virus Genes 35:597-603, 2007.
13. Zhai Y G, Wang H Y, Sun X H, et al. Complete sequence characterization of isolates of Getah virus (genus Alphavirus, family Togaviridae) from China. J Gen Virol 89:1446-1456, 2008.
14. Charlotte Rafaluk, Gunther Jansen, Hinrich Schulenburg, and Gerrit Joop. When experimental selection for virulence leads to loss of virulence. Trends Parasitol. 2015 September; 31 (9): 426-34.
15. José Luis Martínez, Fernando Baquero and Dan I Andersson. Beyond serial passages: new methods for predicting the emergence of resistance to novel antibiotics. Current Opinion in Pharmacology 2011, 11:439-445.

### REFERENCE TO A “SEQUENCE LISTING,” A TABLE, OR A COMPUTER PROGRAM LISTING APPENDIX SUBMITTED AS AN ASCII TEXT FILE

The material in the XML file, named “WANH-65386US1-Sequence-Listing.xml”, created Dec. 19, 2025, file size of 90,112 bytes, is hereby incorporated by reference.

What is claimed:

1. An M1 virus, wherein an amino acid residue corresponding to the 358th site of the NS3 protein of the M1 virus is not M; and/or an amino acid residue corresponding to the 4th site of the envelope protein E2 is not E or K.

2. The M1 virus according to claim 1, wherein the amino acid residue corresponding to the 358th site of the NS3 protein of the M1 virus is: G, A, L, I, V, P, S, Q, T, C, N, F, Y, W, D, E, K, R or H; and/or the amino acid residue corresponding to the 4th site of the envelope protein E2 is: M, G, A, L, I, V, P, S, Q, T, C, N, F, Y, W, D, R or H.

3. The M1 virus according to claim 1, wherein an amino acid sequence of the NS3 protein comprised in the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 8 or SEQ ID NO: 18; and/or an amino acid sequence of the E2 protein comprised in the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 12 or SEQ ID NO: 31.

4. An M1 virus, wherein an amino acid residue corresponding to the 358th site of the NS3 protein encoded by a nucleotide sequence of the M1 virus is not M; and/or an amino acid residue corresponding to the 4th site of the E2 protein is not E or K;
preferably, the nucleotide sequence of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an M1 sequence shown as SEQ ID NO: 5 or SEQ ID NO: 15 or GenBank Accession No. EU015061.1 or GenBank Accession No. EF011023.1 or CCTCC V201423;
preferably, the amino acid residue corresponding to the 358th site of the NS3 protein is: G, A, L, I, V, P, S, Q, T, C, N, F, Y, W, D, E, K, R or H; and/or the amino acid residue corresponding to the 4th site of the E2 protein is: M, G, A, L, I, V, P, S, Q, T, C, N, F, Y, W, D, R or H;
preferably, the M1 virus has a mutation of the amino acid site relative to a wild-type M1 virus or a pseudo-wild-type M1 virus;
preferably, the M1 virus has a mutation of the amino acid site relative to an M1 virus having a sequence shown as SEQ ID NO: 5 or an M1 virus having a sequence shown as SEQ ID NO: 15;
preferably, the mutation is: M358G, M358A, M358L, M358I, M358V, M358P, M358S, M358Q, M358T, M358C, M358N, M358F, M358Y, M358W, M358D, M358E, M358K, M358R or M358H on the NS3 protein; and/or K4M, K4G, K4A, K4L, K4I, K4V, K4P, K4S, K4Q, K4T, K4C, K4N, K4F, K4Y, K4W, K4D, K4R, K4H, E4M, E4G, E4A, E4L, E4I, E4V, E4P, E4S, E4Q, E4T, E4C, E4N, E4F, E4Y, E4W, E4D, E4R or E4H on the E2 protein;
preferably, the M1 virus is obtained by a mutation of an amino acid residue M corresponding to the 358th site of the NS3 protein of the M1 virus having the sequence shown as SEQ ID NO: 5 into: G, A, L, I, V, P, S, Q, T, C, N, F, Y, W, D, E, K, R or H; and/or a mutation of an amino acid residue K at the 4th site of the E2 protein into: M, G, A, L, I, V, P, S, Q, T, C, N, F, Y, W, D, R or H; and
preferably, the amino acid residue M corresponding to the 358th site of the NS3 protein of the M1 virus mutates into L relative to the M1 virus having the sequence shown as SEQ ID NO: 15; and/or the amino acid residue E corresponding to the 4th site of the E2 protein mutates into D.

5. An amino acid sequence corresponding to the NS3 protein of an M1 virus according to claim 1, wherein an amino acid residue corresponding to the 358th site of the amino acid sequence is not M;
preferably, the amino acid residue corresponding to the 358th site is: G, A, L, I, V, P, S, Q, T, C, N, F, Y, W, D, E, K, R or H; and
preferably, the amino acid sequence corresponding to the NS3 protein of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 8 or SEQ ID NO: 18.

6. An amino acid sequence corresponding to the E2 protein of an M1 virus according to claim 1, wherein an amino acid residue corresponding to the 4th site of the amino acid sequence is not E or K;
preferably, the amino acid residue at the 4th site is M, G, A, L, I, V, P, S, Q, T, C, N, F, Y, W, D, R or H; and
preferably, the amino acid sequence corresponding to the E2 protein of the M1 virus has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 12 or SEQ ID NO: 31.

7. A nucleotide sequence for encoding an amino acid sequence according to claim 5.

8. A nucleotide sequence for encoding an amino acid sequence according to claim 6.

9. A vector, comprising a nucleic acid for encoding the E2 protein of an M1 virus, and/or the NS3 protein, wherein an amino acid residue corresponding to the 358th site of the NS3 protein is not M; and an amino acid residue corresponding to the 4th site of the E2 protein is not E or K;
preferably, the amino acid residue corresponding to the 358th site of the NS3 protein is: G, A, L, I, V, P, S, Q, T, C, N, F, Y, W, D, E, K, R or H;
preferably, the amino acid residue corresponding to the 4th site of the E2 protein is: M, G, A, L, I, V, P, S, Q, T, C, N, F, Y, W, D, R or H;
preferably, an amino acid sequence of the NS3 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 8 or SEQ ID NO: 18;
preferably, an amino acid sequence of the E2 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 12 or SEQ ID NO: 31;
preferably, the vector further comprises a coding sequence of the NS1 protein, the NS2 protein, the NS4 protein, the C protein, the E3 protein, the 6K protein, and/or the E1 protein of the M1 virus;
preferably, an amino acid sequence of the NS1 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 6 or SEQ ID NO: 16;
preferably, an amino acid sequence of the NS2 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 7 or SEQ ID NO: 17;
preferably, an amino acid sequence of the NS4 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 9 or SEQ ID NO: 19;
preferably, an amino acid sequence of the C protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 10 or SEQ ID NO: 20;
preferably, an amino acid sequence of the E3 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 11 or SEQ ID NO: 30;
preferably, an amino acid sequence of the 6K protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 13 or SEQ ID NO: 32;
preferably, an amino acid sequence of the E1 protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 14 or SEQ ID NO: 33;
preferably, the vector further comprises exogenous genes relative to the M1 virus;
preferably, the exogenous genes express anti-tumor-associated molecules;
preferably, the vector is selected from viruses;
preferably, the vector is selected from a retrovirus, a Newcastle disease virus, a rabies virus, a vesicular stomatitis virus, a Maraba virus, an alphavirus, a Newcastle disease virus, a reovirus, an adenovirus, an adeno-associated virus, a herpes simplex virus, a vaccinia virus, or a measles virus; and
preferably, the vector is selected from plasmids.

10. A nucleotide sequence comprising an M1 virus according to claim 1.

11. A vector, comprising a nucleotide sequence according to claim 10, wherein preferably, the vector is selected from plasmids.

12. A virus vector, wherein the virus is an M1 virus according to claim 1;
preferably, the vector is inserted with exogenous genes; and
preferably, the exogenous genes express anti-tumor-associated molecules.

13. A method for treating a tumor in a subject in need thereof, comprising: administering to the subject an effective amount of M1 virus according to claim 1.

14. The method according to claim 13, wherein the tumor is selected from a solid tumor and a hematological tumor;
preferably, the solid tumor is selected from one or more of liver cancer, colorectal cancer, bladder cancer, breast cancer, cervical cancer, prostate cancer, a glioma, melanoma, pancreatic cancer, nasopharyngeal carcinoma, lung cancer, stomach cancer, adrenocortical carcinoma, accessory renal cortical carcinoma, anal cancer, appendix cancer, astrocytoma, atypical teratoma, a rhabdoid tumor, basal cell carcinoma, cholangiocarcinoma, bladder cancer, bone cancer, a brain tumor, a bronchial tumor, Burkitt lymphoma, a carcinoid tumor, a heart tumor, cholangiocarcinoma, chordoma, carcinoma of large intestine, craniopharyngioma, ductal carcinoma in situ, a germ tumor, endometrial cancer, ependymoma, esophageal cancer, olfactory neuroblastoma, a germ Cell tumor, an extragonadal germ cell tumor, retinoblastoma, carcinoma of fallopian tube, carcinoma of gallbladder, head and neck cancer, hypopharyngeal cancer, Kaposi's sarcoma, renal carcinoma, Langerhans cell histiocytosis, laryngeal cancer, lip cancer, oral cancer, Merkel cell carcinoma, malignant mesothelioma, multiple endocrine neoplasia syndrome, mycosis fungoides, carcinoma of nasal cavity and nasal sinuses, neuroblastoma, non-small cell lung cancer, ovarian cancer, a pancreatic neuroendocrine tumor, an islet cell tumor, papillomatosis, paraganglioma, carcinoma of nasal sinuses and nasal cavity, parathyroid carcinoma, carcinoma of penis, throat cancer, a pituitary tumor, pleuropulmonary blastoma, primary peritoneal carcinoma, retinoblastoma, a salivary gland tumor, sarcoma, Sezary syndrome, skin cancer, small cell lung cancer, carcinoma of small intestine, soft tissue sarcoma, squamous cell carcinoma, testicular cancer, thymoma and thymic cancer, thyroid cancer, urethral cancer, uterine cancer, endometrium and uterine sarcoma, vaginal cancer, a vascular tumor, vulvar cancer, and solitary myeloma;
preferably, the hematological tumor is selected from one or more of B-cell acute lymphoblastic leukemia (BALL), T-cell acute lymphoblastic leukemia (TALL), acute lymphoblastic leukemia (ALL), chronic myelogenous leukemia (CML), chronic lymphocytic leukemia (CLL), B-cell promyelocytic leukemia, blastic plasmacytoid dendritic cell neoplasm, Burkitt lymphoma, diffuse large B-cell lymphoma, follicular lymphoma, hairy cell leukemia, small cell or large cell-follicular lymphoma, MALT lymphoma, mantle cell lymphoma, marginal zone lymphoma, multiple myeloma, non-Hodgkin lymphoma, plasmablastic lymphoma, plasmacytoid dendritic cell neoplasm, Waldenstrom macroglobulinemia, and pre-leukemia; and
more preferably, the tumor is selected from one or more of liver cancer, colorectal cancer, bladder cancer, breast cancer, cervical cancer, prostate cancer, a glioma, melanoma, pancreatic cancer, nasopharyngeal carcinoma, lung cancer, and stomach cancer.

15. A method for treating a tumor in a subject in need thereof, comprising: administering to the subject an effective amount of the M1 virus according to claim 4.

16. A method for treating a tumor in a subject in need thereof, comprising: administering to the subject an effective amount of nucleotide sequence according to claim 10.

17. A method for treating a tumor in a subject in need thereof, comprising: administering to the subject an effective amount of amino acid sequence of the NS3 protein according to claim 5.

18. A method for treating a tumor in a subject in need thereof, comprising: administering to the subject an effective amount of amino acid sequence of the E2 protein according to claim 6.

19. A method for treating a tumor in a subject in need thereof, comprising: administering to the subject an effective amount of nucleotide sequence according to claim 7.

20. A method for treating a tumor in a subject in need thereof, comprising: administering to the subject an effective amount of nucleotide sequence according to claim 8.

21. A method for treating a tumor in a subject in need thereof, comprising: administering to the subject an effective amount of vector according to claim 12.

22. An anti-tumor agent, comprising an M1 virus according to claim 1.

23. An anti-tumor agent, comprising a vector according to claim 9.

24. An anti-tumor agent, comprising a vector according to claim 11.

25. An anti-tumor agent, comprising a vector according to claim 12.

26. A composition, comprising an effective amount of M1 virus according to claim 1 and a pharmaceutically acceptable carrier, wherein
preferably, the composition further comprises an immune checkpoint inhibitor;
preferably, the composition further comprises a chemotherapeutic agent;
preferably, the composition comprises at least 10¹ virus particles or PFUs;
preferably, the composition comprises 10¹ to 10³⁰ virus particles or PFUS;
preferably, the compositions comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² virus particles or PFUS;
preferably, the composition comprises 2×10⁶ virus particles or PFUs; and
preferably, the composition is used to resist a tumor.

27. A composition, comprising an effective amount of M1 virus according to claim 4 and a pharmaceutically acceptable carrier, wherein
preferably, the composition further comprises an immune checkpoint inhibitor;
preferably, the composition further comprises a chemotherapeutic agent;
preferably, the composition comprises at least 10¹ virus particles or PFUs;
preferably, the composition comprises 10¹ to 10³⁰ virus particles or PFUS;
preferably, the compositions comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² virus particles or PFUs;
preferably, the composition comprises 2×10⁶ virus particles or PFUs; and
preferably, the composition is used to resist a tumor.

28. A composition, comprising a vector according to claim 9 and a pharmaceutically acceptable carrier, wherein
preferably, the composition further comprises an immune checkpoint inhibitor;
preferably, the composition further comprises a chemotherapeutic agent;
preferably, the composition comprises at least 10¹ virus particles or PFUs;
preferably, the composition comprises 10¹ to 10³⁰ virus particles or PFUs;
preferably, the compositions comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² virus particles or PFUS;
preferably, the composition comprises 2×10⁶ virus particles or PFUs; and
preferably, the composition is used to resist a tumor.

29. A composition, comprising a vector according to claim 12 and a pharmaceutically acceptable carrier, wherein
preferably, the composition further comprises an immune checkpoint inhibitor;
preferably, the composition further comprises a chemotherapeutic agent;
preferably, the composition comprises at least 10¹ virus particles or PFUs;
preferably, the composition comprises 10¹ to 10³⁰ virus particles or PFUs;
preferably, the compositions comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² virus particles or PFUs;
preferably, the composition comprises 2×10⁶ virus particles or PFUs; and
preferably, the composition is used to resist a tumor.
